(19) United States
(12) Patent Application Publication
(10) Pub. No.: US 2011/0236501 A1
(43) Pub. Date: Sep. 29, 2011

(54) INJECTABLE DUAL DELIVERY ALLOGRAPH BONE/POLYMER COMPOSITE FOR TREATMENT OF OPEN FRACTURES

(75) Inventors: Scott A. Guelcher, Franklin, TN (US); Bing Li, Troy, NY (US); Andrea E. Hafeman, Los Angeles, CA (US); Joseph C. Wenke, LaVernia, TX (US); Kate V. Brown, Raolett (GB)

(73) Assignee: Vanderbilt University

(21) Appl. No.: 13/005,481

(22) Filed: Jan. 12, 2011

Related U.S. Application Data
(63) Continuation-in-part of application No. 12/676,710, filed on Nov. 5, 2010.
(60) Provisional application No. 60/970,194, filed on Sep. 5, 2007.

(51) Int. Cl. A61K 35/32; A61P 19/00; A61P 35/00; A61P 31/00; A61K 31/7036; A61P 17/02; A61K 38/18

(52) U.S. Cl. 424/549; 514/40; 514/8.2; 514/8.8

(57) ABSTRACT

A biodegradable polyurethane scaffold, comprising at least one polyisocyante, polyisocyanate prepolymer, or both, at least one polyester polyol, at least one catalyst, wherein the density of said scaffold is from about 50 to about 250 kg m-3 and the porosity of the scaffold is greater than about 70 (vol %) and at least 50% of the pores are interconnected with another pore, and wherein the scaffold incorporates at least one biologically active component in powder form.

### PRIORITY

This application is a continuation-in-part of U.S. patent application Ser. No. 12/676,710 filed Mar. 5, 2010 which claims priority to International Application No. PCT/US2008/075481 filed on Sep. 5, 2008 which claims priority under 35 U.S.C. §119(e) of Provisional Patent Application No. 60/970,194 filed on Sep. 5, 2007 and Provisional Patent Application No. 61/294,481 filed Jan. 12, 2010. The content of these applications are incorporated herein by reference in their entirety.

### GOVERNMENT SUPPORT

This invention was made with support from the US Army Institute for Surgical Research grant number DOD-W81XWH-06-1-0654 and the Orthopaedic Trauma Research Program grant number DOD-W81XWH-07-1-0211. The United States Government has rights to this invention.

### BACKGROUND AND SUMMARY OF THE INVENTION

Bone regeneration is required for healing of open fractures, and healing is often complicated by chronic infection. Restoration of bone form and function is achieved through the physiological and regenerative process of bone healing. Infection is a significant clinical problem in bone fracture healing, especially for open fractures with large gaps in the bone which happens frequently in combat-related trauma, for example. Current approaches require a two-step process, in which the infection is first controlled by implantation of non-degradable tobramycin-impregnated PMMA beads, followed by implantation of a bone graft to promote bone healing. To reduce the healing time of the patient, it is desirable to promote bone fracture healing and control infection through one surgical procedure.

Biodegradable polymers have been used extensively as scaffolds to support tissue regeneration. Ideally, scaffolds should possess a three dimensional structure, high porosity with an interconnected pore structure, and a suitable surface structure for cells. Polyurethanes (PUR) have been investigated as scaffolds in bone tissue engineering due to their porous, biodegradable, and biocompatible properties. PUR scaffolds support attachment, growth, and differentiation of osteoprogenitor cells in vitro, and biodegrade to nontoxic products in vivo. Moreover, the physical and biological properties, as well as the degradation rate, of PUR scaffolds can be tuned to targeted values through the choice of intermediates used in the synthesis. Therefore, compared with currently available scaffolds and delivery systems, PUR scaffolds can offer many advantages in the design of injectable and biodegradable polymer compositions.

The recent development of injectable, biodegradable, and in situ cross-linkable biomaterials seek to alleviate many of the challenges associated with current surgical techniques and prefabricated tissue engineered implants. PUR scaffolds can be used as injectables through a two-component liquid system which cures in situ to form a solid providing a strong bond with surrounding tissues due to the following advantages. Firstly, the moderate exothermal polymerization process does not cause detrimental effects to the surrounding tissue. Secondly, the mechanical and physical properties can be tuned according to selected applications. Thirdly, the resulting polymer scaffolds allow for diffusion of nutrients, providing a cytocompatible environment and guiding cell attachment, growth, and differentiation. The scaffolds of the present invention also serve as a delivery device for drugs which promote cell infiltration and tissue remodeling. Based on the functional mechanisms of different drugs, the release profiles of them from PUR scaffolds can be controlled through adopting various including strategies. Dual release can also be achieved through embedding two different drugs in the same scaffold.

Embodiments of the present invention relate to the delivery of biologically active agents from biodegradable polyurethane scaffolds. In one embodiment, the biologically active components are incorporated as a labile powder in one of the components of the reactive polyurethane prior to mixing. Previous studies have shown that biologically active proteins with hydroxyl groups and amines, such as proteins, covalently bind to the polyurethane when dissolved in solution. For example, release of ascorbic acid from biodegradable polyurethane foams has been reported (Beckman, WO2004065450, incorporated herein by reference). However, as disclosed herein, when the protein is dissolved in solution, the cumulative release after 20 days is low (<20%). With embodiments of the present invention, substantially higher (>60%) cumulative release of the biological can be achieved. We have demonstrated release, as well as in vitro and in vivo bioactivity, for a variety of biologicals, including tobramycin (antibiotic), colistin, BSA, PDGF, and BMP-2. All of these biologicals have hydroxyl groups and amines, and yet they achieve a high cumulative release.

Bacteria in a open fracture wound, which can cause osteomyelitis and compromise fracture healing. Such contamination should be treated immediately to allow proper healing. Local delivery of antibiotics can achieve high local concentrations while systemic levels remain low. This approach is a common clinical practice and has been demonstrated in animal studies to be safe and effective for treating osteomyelitis.

While local antibiotic delivery from PMMA beads is an established clinical treatment of infected fractures, surgical removal of the beads is required before implanting a bone graft. A more ideal therapy would comprise a scaffold and antibiotic delivery system administered in one procedure. Biodegradable polyurethane scaffolds have been shown in previous studies to promote new bone formation in vivo, but their potential to control infection through release of antibiotics has not been investigated.

Additionally, local delivery of tobramycin from implanted poly(methyl methacrylate) (PMMA) cement beads is an established therapy for treating infected fractures, but only a small amount (<10%) of the drug is released. Tobramycin is a known antibiotic drug. See, for example, The Merck Index, Twelfth Edition, page 1619.

As indicated above, the PMMA beads are not resorbable and must be surgically removed after two to six weeks, at which time a bone graft is implanted to aid healing.

A much needed therapy for bone infections such as those described above would include both a delivery system and a scaffold to promote fracture healing. Preferably, the system would release the antibiotic dose over an extended period of time, biodegrade to non-cytotoxic decomposition products at a rate comparable to that of tissue healing, and support ingrowth of cells and new tissue.

Thus, the tissue engineered scaffolds of the present invention offer advantages for controlled release of bioactive materials, including antibiotics for example, by providing both sustained release of the bioactive component as well as a template for infiltration of new cells and tissue.

Scaffolds synthesized from two-component polyurethanes have been shown to degrade to non-cytotoxic decomposition products and to support the ingrowth of cells and new tissue in vitro and in vivo. By varying the composition, scaffolds with tunable mechanical properties ranging from soft elastomers to leathery and glassy plastics have been prepared. Additionally, biodegradable polyurethane scaffolds prepared from linear segmented elastomers were shown to support controlled release of bFGF, suggesting the potential utility of polyurethane scaffolds for drug delivery applications.

Embodiments of the present invention include novel methods of incorporating a bioactive element, such as an antibiotic into a reactive polyurethane (PUR) scaffold. The effects of scaffold hydrophilicity and degradation rate on tobramycin release and bioactivity were investigated, as well as the effects of tobramycin on the dynamic mechanical properties of the polyurethane scaffolds.

Another embodiment of the present invention is a method of delivering a bioactive agent to a wound site, including a bone fracture site, using injectable, biodegradable polyurethane foams. These materials support osteoblast cell migration and proliferation, and degrade to non-cytotoxic decomposition products. Polyurethane (PUR) scaffolds have also been shown to promote ingrowth of new cancellous bone when implanted in the iliac crest of sheep.

Embodiments of the present invention include PUR scaffolds to release antibiotics using at least two approaches: (1) incorporation as a powder, and (2) microencapsulation in PLGA microspheres. These biomaterials present potential clinical opportunities for treatment of various indications, including osteomyelitis.

Aspects of the present invention relate to methods and compositions for treatment of bone fractures. Specific embodiments of the present invention include products and methods related to materials that are injectable, biodegradable, and undergo controlled degradation and release of bioactive components. Scaffold degradation and release of bioactive components can be controlled independently. Conventional materials, such as tricalcium phosphates, polymethyl methacrylate, and poly(D,L-lactide-co-glycolide) cannot meet all of these performance requirements.

Scaffolds of the present invention may be both biodegradable and resorbable, so they can minimize total surgery time and invasiveness for patients. Furthermore, PMMA bone cement only delivers approximately 2-5% of its encapsulated tobramycin, while scaffolds of the present invention have a 50-90% delivery efficiency rate. A great benefit of the reactive liquid molding synthesis of our scaffolds is that it allows them to be injectable and therefore minimally invasive during implantation. In addition, they can expand to fill the contours of the fracture site, enhancing bone-scaffold contact and fixation.

Embodiments of the present invention offer injectable polyurethane scaffolds incorporating tobramycin were prepared by reactive liquid molding. Scaffolds had compressive moduli of 15-115 kPa and porosities ranging from 85-93%. Tobramycin release was characterized by a 45-95% burst (tuned by the addition of PEG), followed by up to 2 weeks of sustained release, with total release 4-5 times greater than equivalent volumes of PMMA beads. Released tobramycin remained biologically active against S. aureus, as verified by Kirby-Bauer and time-kill assays. Similar results were observed for the antibiotics colistin and tigecycline. The versatility of the present invention, as well as their potential for injection and controlled release, may present promising opportunities for new therapies for healing of infected wounds.

Thus, embodiments of the present invention include biodegradable polyurethane scaffolds that comprise at least one polyisocyante, polyisocyanate prepolymer, or both; at least one polyester polyol; and at least one catalyst. The density of said scaffold is from about 50 to about 250 kg m-3 and the porosity of the scaffold is greater than about 70 (vol %) and at least 50% of the pores are interconnected with another pore; and the scaffold incorporates at least one biologically active component in powder form. The biologically active component may have a hydroxyl or amine group. Additionally, the biologically active component may be at least one antibiotic, protein, anti-cancer agent, or combinations thereof. Examples include at least one of tobramycin, colistin, tigecycline, BSA, PDGF, BMP-2. In embodiments of the invention, the biologically active component is in powder, including a labile powder.

In other embodiments of the present invention, the polyisocyanate is an aliphatic polyisocyanate. Examples include lysine methyl ester diisocyanate (LDI), lysine triisocyanate (LTI), 1,4-diisocyanatobutane (BDI), and hexamethylene diisocyanate (HDI), and dimers and trimers of HDI.

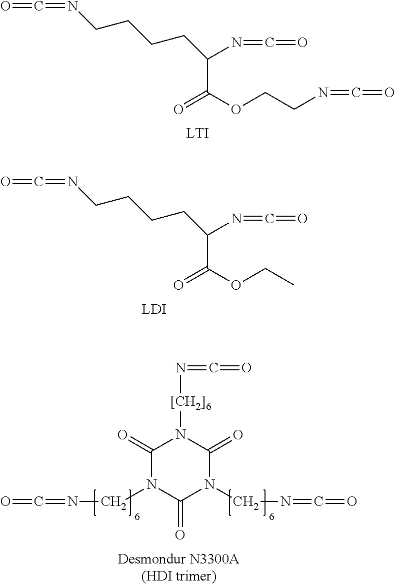

In embodiments of the present invention the biologically active agent is present in an amount of from about 2 to about 10 wt %; or the biologically active agent is present in an amount of from 4 to about 10 wt %. When the biologically active agent is an antibiotic, it may be present in an amount, for example, of from about 1-12 wt %. As another example, when the biologically active agent is a protein, it may be present in an amount of from about 0.01 to about 10000 μg/ml of scaffold; or in an amount from about 0.1 to about 5000 μg/ml of scaffold; or in an amount from about 1 to about 5000 μg/ml of scaffold.

Additional embodiments of the present invention include compositions that comprises materials of the scaffolds described herein. One aspect is a composition that comprises at least one polyisocyante, polyisocyanate prepolymer, or both; at least one polyester polyol; at least one catalyst; and at least one biologically active component in powder form. The biological agents are described above.

Additional embodiments include methods of using the compositions and scaffolds of the present invention. One example is their use in a method of delivering a biologically active agent to a would site. This example can comprise providing a composition that comprises at least one polyisocyante, polyisocyanate prepolymer, or both; at least one polyester polyol; at least one catalyst; and at least one biologically active component in powder form; and contacting the composition with a wound site. The wound site may be, for example, part of a bone or skin.

As another embodiment of the present invention relates to infection being a significant clinical problem in bone wound healing (1), even in the presence of an osteogenic factor, especially for open fractures with large gaps in the bone which happens frequently in combat-related trauma. Present clinical approaches require a two-step process, in which the infection is first controlled by implantation of non-degradable antibiotic-impregnated PMMA beads, followed by implantation of a bone graft to promote bone healing. To reduce the healing time of the patient, it is desirable to promote bone fracture healing and control infection through one surgical procedure. The present inventors have shown that sustained delivery of BMP-2 from polyurethane (PUR) was able to promote new bone formation in rat femora (2). Other embodiments relate to the delivery of vancomycin free base (V-FB) from PUR for up to 8 weeks at a concentration well above minimum inhibitory concentration (MIC) which inhibited infection in contaminated rat femoral segmental defect (submitted work). The purpose of the present study is to incorporate both drugs in the same PUR scaffold, and to test the composite's ability of controlling infection and promoting bone wound healing simultaneously.

Other embodiments will be apparent from a reading of the disclosure.

### BRIEF DESCRIPTION OF THE DRAWINGS

FIG. 1 shows an example of the delivery of an embodiment of the present invention.

FIG. 2 is a graph showing tobramycin release kinetics.

FIG. 3 shows a rat would healing model.

FIG. 4 is graph showing tobramycin release.

FIG. 5 shows in vivo response to foam in a rat excisional dermal wound.

FIG. 6 is a scanning electron micrograph (SEM) of T6C3G1L-PEG0 scaffold.

FIG. 7 shows in vitro tobramycin release from PUR scaffolds and PMMA beads. Materials were incubated at 37° C. in PBS, which was completely removed and refreshed at each time point. Tobramycin concentration in the releasate was measured by HPLC.

FIG. 8 shows zones of inhibition (ZI) measured after 24 hours for PUR scaffolds using the Kirby-Bauer test. PMMA control: ˜6-mm PMMA beads with 4.0 wt-% tobramycin. Positive control: 10 μg tobramycin BBL SensiDiscs. Negative control: PUR scaffolds with no tobramycin. Asterisks denote statistical significance (p<0.005) with respect to the positive control and PMMA.

FIG. 9 shows bioactivity of tobramycin released from PUR scaffolds after 8, 20, and 30 days of incubation in PBS, evaluated by Kirby-Bauer tests. Blank BBL SensiDiscs were loaded with 0.5 μg tobramycin (in 10 μL) PBS) from each releasate (as determined by HPLC), as well as 0.5 μg exogenous tobramycin for the positive control. Asterisks denote statistical significance (p<0.005) with respect to the positive control.

FIG. 10 shows storage (bold) and loss moduli as a function of shear rate in compression mode during DMA frequency sweeps from 0.1 to 10 Hz. Illustrated are the results from T6C3G1L scaffolds with (A) 0%, (B) 30%, and (C) 50% PEG, each with (solid line) and without (dotted line) tobramycin.

FIG. 11 shows DMA stress relaxation response to 2% strain (compression) over 20 minutes of PUR scaffolds with (A) 0%, (B) 30%, and (C) 50% PEG, with (solid line) and without (dotted line) tobramycin.

FIG. 12 is a chart that shows in vitro release profile of BSA-FITC from PUR scaffold. BSA-FITC was included into the scaffold as solution in presence of 0.5% glucose, and as powder in presence of different weight percentage of glucose.

FIG. 13 is a chart that shows in vitro release profile of PDGF-BB from PUR scaffold including PDGF-BB powder (PUR-PDGF). Also included are 0.05% heparin and 2% glucose, and the release kinetics was determined by lodine125 labeling and ELISA respectively.

FIG. 14 is a chart that shows in vitro release profile of PDGF-BB from PLGA particles, granules and polyurethane scaffold containing granules (PUR-Granules). The release kinetics was determined by lodine125 labeling (A) and ELILSA (B) respectively.

FIG. 15 is a chart that shows in vitro cell proliferation ability of PDGF-BB releasates from PUR-PDGF (A), Particles (B), Granules (C), and PUR-Granules (D) respectively.

FIG. 16 is scanning electronic microscopic images of polyurethace scaffold containing 2% glucose (A), and containing 15% granules (B).

FIG. 17 is scanning electronic microscopic images of polyurethace scaffold containing 80 um PLGA particles (A), and 1 um PLGA particles (B).

FIG. 18 shows data in connection with the release of BSA-FITC from PUR scaffolds.

FIG. 19 shows data in connection with the release of BMP-2 from PUR scaffolds.

FIG. 20 shows the results of an ALP assay of BMP-2 releasate liquids.

FIG. 21 shows a photograph of a 6 mm segmental defect in a rat femor stabilized by a polyacetal plate.

FIG. 22 is a chart that shows bacterial counts measured in soft tissue after 14 days of treatment. The groups represented include no treatment, PMMA beads, PUR scaffords with no PEG content (PEGO), and PUR scaffolds with 20% PEG content (PEG 20).

FIG. 23 is a chart that shows rhBMP-2 release kinetics measured by ELISA for 24 days.

FIG. 24 is a chart that shows alkaline phosphatase (ALP) measured for MC3T3 cells cultured on tissue culture polystyrene in the presence of rhBMP-2 released from PUR scaffolds.

FIG. 25 shows the data in connection with the in vitro release of BMP 2 and V-FB from PUR.

FIG. 26 shows photographs of a test of PUR composite in contaminated critical sized rat femoral segmental defect.

FIG. 27 shows data as the percentage of rats with signs of infection at week eight for the various treatment groups indicated.

FIG. 28 shows photographs of Injectable PUR/allograft composites, where (A) shows injection of composites into a 15-mm rabbit calvarial defect, (B) shows a radiograph after 6 weeks shows new bone formation around the perimeter of the defect (dashed line), and (C) shows a histological section (H&E stain) of the defect taken from a rabbit sacrificed on day 12 post-surgery. No adverse tissue response was observed, and new bone formation was observed at the edge of the defect.

FIG. 29 shows photographs of clinical applications of Plexur™ P, where (a) shows possible shapes and sizes of Plexur™ P, (b) shows the hydrophilic nature of Plexur™ P that absorbs blood during surgery, (c) shows a pre-operative x-ray of a persistent non-union in an older female patient showing the gap in the bone, (d) shows Plexur™ P inserted in the non-union site to stabilize the defect and to establish an osteoconductive pathway, (e) shows a post-operative x-ray at 6 months shows healing of the non-union, (f) shows an infected non-union of the clavicle, and (g) shows a post-operative x-ray shows complete healing with no infection after 1 year.

FIG. 30 shows photographs of clinical applications of Plexur™ M, where (a) shows Plexur™ M (20 cc) in a commercial package, (b) shows Plexur™ M in various stages of manipulation, (c) shows Plexur™ M implanted in a debrided calcaneous fracture, (d) shows a 6-week radiograph depicting new bone ingrowth into the defect, (e) shows Plexur™ M implanted in a tumor osteoid osteoma in the proximal tibia, and (1) shows a 7-week radiograph remodeling of the graft and integration with host bone.

FIG. 31 shows an example of a dual-delivery device for vancomycin (V) and rhBMP2 of an embodiment of the present invention, where (A) shows the conceptual design, (B) shows a representative radiograph of collagen+rhBMP2 (standard of care) implants showing a non-union after 8 weeks post-implantation in infected rat femoral 6-mm defects, and (C) shows a representative radiograph of a PUR+V+rhBMP2 implant showing robust new bone formation after 8 weeks post-implantation in infected rat femoral 6-mm defects.

FIG. 32 shows photographs of thin decalcified sections of Plexur™ LV injected in bilateral femoral plug defects in rats stained with fuchsin red-toluidene blue. (A)-(C) show low magnification images showing host bone (“HB”, pink), residual polymer (“P”, red), allograft particles embedded in polymer that have not been resorbed (“A”, pink), regions of active remodeling (labeled “RM”, purple) into the interior of the composite, osteoid (“O”, blue/purple), and bone marrow (“BM”, blue/purple) around the surface of the material, where (A) corresponds to the case where the wound was closed immediately after injection of the material, and (B) and (C) correspond to the case where the wound was closed 15 minutes after injection. (D)-(F) show higher magnification views of the implant shown in (C). (G)-(H) show higher magnification of regions of active remodeling characterized by allograft (pink) resorption, cells (purple), and collagen deposition (blue), where (G) shows the cellular pathway in an interior region of the composite, and (H) shows the infiltration of cells into the composite from the bone marrow where in the center of the image there is an allograft particle undergoing active remodeling that appears to be embedded in polymer except for a small breach (labeled “B”) where cells infiltrated along the allograft/polymer interface.

FIG. 33 shows data and an image of rhBMP2 released from PUR scaffolds embodiments enhancing new bone formation in a rat segmental defect model, where (A) shows data of release kinetics of rhBMP2 for PUR scaffolds, (B) shows an μCT image of a fast release PUR/rhBMP2 scaffold, and (C) shows data Bone volume measured by μCT, where blue (left bar) corresponds to four weeks and red (right bar) corresponds to 8 weeks, and letters represent significant differences.

FIG. 34 shows faxitron images of embodiments of scaffolds in accordance with the present invention implanted in infected rat segmental femoral defects after 8 weeks in which 60 μg/ml (low) and 600 μg/ml (high) rhBMP2 doses were investigated, where the embodiments depicted are as follows: (A) Collagen+low rhBMP2, (B) Collagen+high rhBMP2, (C) PUR+low rhBMP2, (D) PUR+high rhBMP2, (E) PUR+low rhBMP2+vancomycin, and (F) PUR+high rhBMP2 +vancomycin.

### DESCRIPTION OF THE INVENTION

Aspects of the present invention include injectable, biodegradable poly(ester urethane)urea (PEUUR) foams for use as scaffolds and delivery systems for bioactive agents to promote fracture healing and bone regeneration.

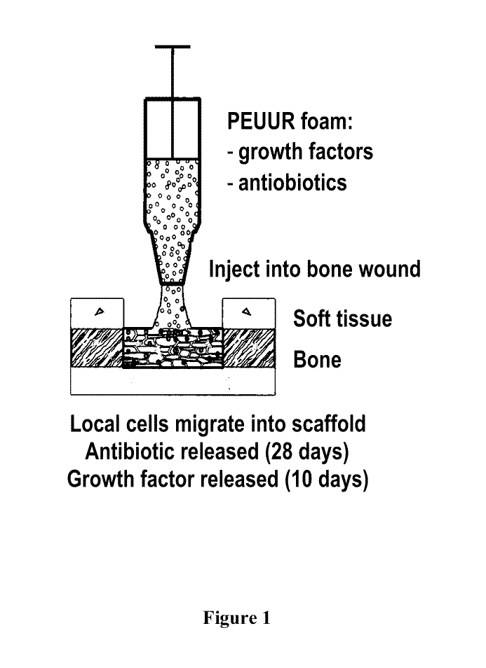

An example of the foam scaffold may be made by reactive liquid molding of two components: an aliphatic isocyanate and a resin composed of a poly(c-caprolactone-co-glycolide-co-lactide) polyol, water, triethylenediamine catalyst, sulfated castor oil stabilizer, and calcium stearate pore opener. As shown in FIG. 1, an advantage of these materials is that the degradation rate of the scaffold and the bioactive release kinetics can be controlled independently. In addition to providing structural support for healing bone, the scaffolds can locally release bioactive agents to the fracture site at a controlled rate. Such bioactive agents include small molecules (e.g., antibiotics and statins) and proteins, such as bone morphogenetic protein-2 (BMP-2) and platelet-derived growth factor (PDGF). The antibiotics, such as tobramycin, serve to fight infections that can hinder the healing process. Statins have been shown to enhance bone healing by upregulating BMP-2 expression.

Another example of a scaffold of the present invention is a scaffold of Patent Application Publication Number 2007/299151, incorporated herein by reference. Accordingly, an embodiment of the present invention is a scaffold synthesized from the steps of: coating a biodegradable and bioactive polyurethane polymer with human osteoblastic precursor cells, the polymer being synthesized by reacting isocyanate groups of at least one multifunctional isocyanate compound with at least one bioactive agent having at least one reactive group —X which is a hydroxyl group (—OH) or an amine group (—NH₂), the polyurethane being biodegradable within a living organism to biocompatible degradation products including the bioactive agent, the released bioactive agent affecting at least one of biological activity or chemical activity in the host organism; and culturing the osteoblastic precursor cells under conditions suitable to promote cell growth.

Another example of a scaffold of the present invention is the two-component network scaffolds disclosed in Published PCT international application WO 2006/055261, the disclosure of which is incorporated herein by reference. Disclosed herein are methods of synthesizing biocompatible and biodegradable polyurethane foam includes the steps of: mixing at least one biocompatible polyol, water, at least one stabilizer, and at least one cell opener, to form a resin mix; contacting the resin mix with at least one polyisocyanate to form a reactive liquid mixture; and reacting the reactive liquid mixture form a polyurethane foam. The polyurethane foam is preferably biodegradable within a living organism to biocompatible degradation products. At least one biologically active molecule having at least one active hydrogen can be added to form the resin mix.

These porous PUR scaffolds prepared from lysine-derived and aliphatic polyisocyanates by reactive liquid molding have been reported to degrade to non-toxic decomposition products, while supporting the migration of cells and ingrowth of new tissue in vitro and in vivo. However, many polyisocyanates are toxic by inhalation, and therefore polyisocyanates with a high vapor pressure at room temperature, such as toluene diisocyanate (TDI, 0.018 mm Hg) and hexamethylene diisocyanate (HDI, 0.05 mm Hg), may not be suitable for injection in a clinical environment. To overcome this limitation, the present inventors have formulated injectable PUR biomaterials using lysine diisocyanate, a lysine-derived polyisocyanate with a vapor pressure substantially less than that of HDI. However, two-component polyurethanes prepared from LDI exhibit microphase-mixed behavior, which inhibits the formation of hydrogen bonds between hard segments in adjacent chains and may adversely affect mechanical properties.

Other examples include the scaffolds of U.S. patent application Ser. No. 12,195,265, the contents of which are incorporated herein by reference. These aspects of the present invention include biocompatible and biodegradable polyurethane scaffolds made from the steps of: mixing at least one biocompatible polyol, water, at least one stabilizer, and at least one cell opener, to form a resin mix; contacting the resin mix with at least one polyisocyanate to form a reactive liquid mixture; and reacting the reactive liquid mixture form a polyurethane foam.

In aspects, of the present invention, the polyisocyanate is a tri-functional isocyanate. In other aspects of the invention, the resin mix comprises polyethylene glycol.

To promote transport of cells, fluids, and signaling molecules, the foams of the present invention can have a porosity greater than 50 vol-%. The porosity c, or void fraction, is calculated as shown in WO '261, cited above.

In several embodiments, at least one catalyst is added to form the resin mix. Preferably, the catalyst is non-toxic (in a concentration that may remain in the polymer).

The catalyst can, for example, be present in the resin mix in a concentration in the range of approximately 0.5 to 5 parts per hundred parts polyol and, preferably in the range of approximately 1 to 5. The catalyst also can, for example, be an organometallic compound or a tertiary amine compound. In several embodiments the catalyst includes stannous octoate, an organobismuth compound, triethylene diamine, bis(dimethylaminoethyl)ether, or dimethylethanolamine. An example of a preferred catalyst is triethylene diamine.

In several embodiments, the polyol is biocompatible and has a hydroxyl number in the range of approximately 50 to 1600. The polyol can, for example, be a biocompatible and polyether polyol or a biocompatible polyester polyol. In several embodiments, the polyol is a polyester polyol synthesized from at least one of ε-caprolactone, glycolide, or DL-lactide.

Water can, for example, be present in the resin mix in a concentration in a range of approximately 0.1 to 4 parts per hundred parts polyol.

The stabilizer is preferably nontoxic (in a concentration remaining in the polyurethane foam) and can include non-ionic surfactant or an anionic surfactant. The stabilizer can, for example, be a polyethersiloxane, a salt of a fatty sulfonic acid or a salt of a fatty acid, hi the case that the stabilizer is a polyethersiloxane, the concentration of polyethersiloxane in the resin mix can, for example, be in the range of approximately 0.25 to 4 parts per hundred polyol. In the case that the stabilizer is a salt of a fatty sulfonic acid, the concentration of the salt of the fatty sulfonic acid in the resin mix is in the range of approximately 0.5 to 5 parts per hundred polyol. In the case that the stabilizer is a salt of a fatty acid, the concentration of the salt of the fatty acid in the resin mix is in the range of approximately 0.5 to 5 parts per hundred polyol. Polyethersiloxane stabilizer are preferably hydrolyzable. Examples of suitable stabilizers include a sulfated castor oil or sodium ricinoleicsulfonate.

The cell opener, or pore opener, is preferably nontoxic (in a concentration remaining in the polyurethane) and comprises a divalent metal salt of a long-chain fatty acid having from about 1-22 carbon atoms. The cell opener can, for example, include a metal salt of stearic acid. The concentration of the cell opener in the resin mix is preferably in the range of approximately 0.5 to 7 parts per hundred polyol.

The polyisocyanate can, for example, be a biocompatible aliphatic polyisocyanatc derived from a biocompatible polyamine compound (for example, amino acids). Examples of suitable aliphatic polyisocyanates include lysine methyl ester diisocyanate, lysine triisocyanate, 1,4-diisocyanatobutane, or hexamethylene diisocyanate. As stated above, embodiments of the present invention comprises tri-functional isocyanate.

The index of the foam, is defined as:

INDEX=100×number of NCO equivalents/number of OH equivalents

and can be in the range of approximately 80 to 140.

The polyurethane foams of the present invention are preferably synthesized without aromatic isocyanate compounds. The method of the present invention can also include the step of placing the reactive liquid mixture in a mold in which the reactive liquid mixture is reacted to form the polyurethane foam.

In another aspect, the present invention provides a biocompatible and biodegradable polyurethane synthesized via the steps of: mixing at least one polyol, PEG, water, at least one stabilizer, and at least one cell opener; contacting the resin mix with at least one triisocyanate to form a reactive liquid mixture; and reacting the reactive liquid mixture to form a polyurethane foam. The polyurethane foam is preferably biodegradable within a living organism to biocompatible degradation products. At least one catalyst, as described above, can be added to form the resin mix. As also described above, at least one biologically active molecule having at least one active hydrogen can be added to form the resin mix.

In another aspect, the present invention provides method of synthesis of a biocompatible and biodegradable polyurethane foam including the steps of: reacting at least one polyol and PEG with at least one triisocyanate to form an isocyanate-terminated prepolymer; mixing water, at least one stabilizer, at least one cell opener and at least one polyol to form a resin mix; contacting the resin mix with the prepolymer to form a reactive liquid mixture; and reacting the reactive liquid mixture to form a polyurethane foam. At least one catalyst, as described above, can he added to form the resin mix. As also described above, at least one biologically active molecule having at least one active hydrogen can be added to form the resin mix.

In embodiments of the invention, porous scaffolds were synthesized by a one-shot foaming process, allowing for time to manipulate and inject the polymer, followed by rapid foaming and setting.

Other examples of a foam of the present invention are set forth in the Examples, below.

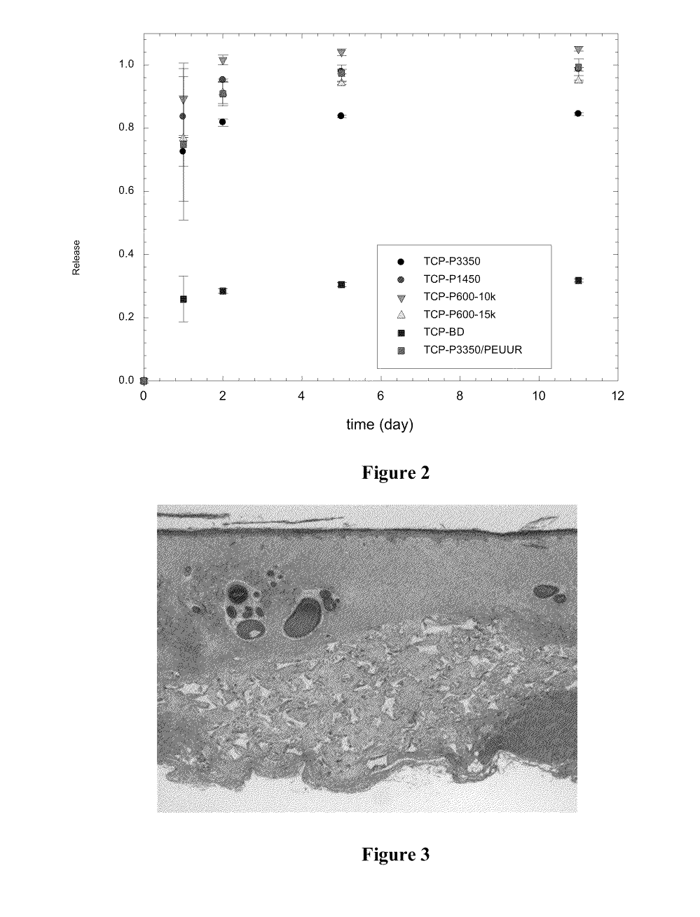

Examples of delivery methods of the present invention include any one or a combination of the following three approaches: direct integration of the agent (in powder form) into the foam formulation; encapsulation into poly(lactic acid-co-glycolic acid) (PLGA) microparticles; and encapsulation in a polyester polyol, which is in turn coated onto tricalcium phosphate (TCP) particles. TCP is an osteoconductive substrate, so its incorporation is beneficial in addition to being a delivery vehicle. In vitro release experiments have shown >80% release during the first three days with the first strategy. The microparticles have exhibited 30% release in twenty days, although this can be altered with the PLGA ratios. As shown in FIG. 2, the last strategy ranges from 30% to 95% delivery within seven days, depending on the composition of the polyol. In order to produce the most effective overall delivery of antibiotics or growth factors, embodiments of the present invention include a combined strategy of the first approach, for an immediate dosage, and either the second or third approach for extended release. In vitro experiments have demonstrated that these scaffolds are not cytotoxic, and the they facilitate cell infiltration, proliferation, and differentiation. In vivo, implantation in a rat wound healing model have shown integration into the surrounding tissue, efficient wound healing, production of new collagen matrix, and biodegradation of the material, with minimal inflammatory response.

As indicated above, biologically active agents can optionally be added to the resin mix. Typically, the biodegradable compounds of the present invention degrade by hydrolysis. As used herein, the term “biocompatible” refers to compounds that do not produce a toxic, injurious, or immunological response to living tissue (or to compounds that produce only an insubstantial toxic, injurious, or immunological response). The term nontoxic as used herein generally refers to substances or concentrations of substances that do not cause, either acutely or chronically, substantial damage to living tissue, impairment of the central nervous system, severe illness, or death. Components can be incorporated in nontoxic concentrations innocuously and without harm. As used herein, the term “biodegradable” refers generally to the capability of being broken down in the normal functioning of living organisms/tissue (preferably, into innocuous, nontoxic or biocompatible products).

Examples of bioactive agents of the present invention include synthetic molecules, biomolecules, or multimolecular entities and include, but are not limited to, enzymes, organic catalysts, ribozymes, organometallics, proteins, glycoproteins, peptides, polyamino acids, antibodies, nucleic acids, steroidal molecules, antibiotics, antivirals, antimycotics, anticancer agents, analgesic agents, antirejection agents, immunosuppressants, cytokines, carbohydrates, oleophobics, lipids, extracellular matrix and/or its individual components, demineralized bone matrix, pharmaceuticals, chemotherapeutics, and therapeutics. Cells and non-cellular biological entities, such as viruses, virenos, virus vectors, and prions can also be bioactive agents. Biologically active agents with at least one active hydrogen are preferred. Examples of chemical moieties with an active hydrogen are amine and hydroxyl groups. The active hydrogen reacts with free isocyanate in the reactive liquid mixture to form a covalent bond (e.g., urethane or urea linkage) between the bioactive molecule and the polyurethane. As the polyurethane degrades, the bioactive molecules are released and are free to elicit or modulate biological activity. The incorporation of biologically active components into biocompatible and biodegradable polyurethanes is discussed in some detail in US Patent Application No. 2005/0013793 (U.S. patent application Ser. No. 10/759,904).

One example of an agent that can be used in connection with the present invention is poly (lactic acid) (PLA), poly (glycolic acid) (PGA), and especially their copolymers (e.g., poly (lactic-co-glycolic acid), PLGA) are among the most commonly used family of biodegradable polymers. The drug release profile from PLGA microspheres is controlled by many factors, such as molecular weight, hydrophilicity, morphology, and size etc. Therefore, PLGA microspheres are also tunable delivery vehicles. One strategy which has been used extensively is to include poly(ethylene glycol) (PEG) segments which can enhance the release profile by modification of the PLGA microspheres' morphology such as improving the porosity and internal hydrophilicity. However, PLGA microspheres do not provide a template for ingrowth of new tissue. Incorporating the microspheres in a PUR scaffold yields an injectable composite biomaterial that accomplishes both controlled release of biologicals, as well as a template for cell infiltration and tissue ingrowth.

Another example is bone morphogenetic protein-2 (BMP-2). Growth factors are polypeptides that transmit signals to modulate cellular activities. They regulate cellular proliferation, differentiation, migration, adhesion, and gene expression. Bone morphogenetic proteins (BMPs) are currently attracting the most corporate and clinical interest, and the osteoinductive capacity of BMP-2 has been demonstrated in preclinical models and evaluated in clinical trials. Many of the animal models used to evaluate the capacity of BMP-2 to heal bone defects have utilized critical-size defects, and healing of long bone critical-size defects by BMP-2 has been demonstrated in species including rats, rabbits, dogs, sheep and non-human primates. Systemic administration of rhBMP-2 increases mesenchymal stem cell activity and reverses ovariectomy-induced and age-related bone loss in two different mouse models, indicating that BMP-2 may be utilized for the treatment of osteoporosis. Recent studies show that rhBMP-2 delivered in an injectable formula with a calcium phosphate carrier or with a liposome carrier accelerates bone healing in a rabbit ulna osteotomy model and a rat femur bone defect model. BMP-2 is shown to be efficacious in several fusion applications, including intervertebral and lumbar posterolateral fusion. BMP-2 has also been shown to induce new dentine formation and BMP-2 is an effective bone inducer around dental implants for periodontal reconstruction.

Recombinant human BMP-2 delivered in a collagen sponge is an FDA approved therapeutic for posterior-lateral spine fusion (InFuse-Sofamor/Danek-Medtronic). However, due to the fact that BMP-2 is a morphogen and functions in later stages of cell growth to promote cell differentiation into osteoblasts, long-term release is desired to achieve an ideal effect in promoting bone fracture healing. Injection of BMP-2 in a calcium phosphate carrier at one or two weeks after surgery which has a BMP-2 retention period of up to 6 weeks, is more effective than injection within one day to enhance osteotomy-site healing in primates. This is the reason that achieving a sustainable release of BMP-2 for at least 30 days is one of the biggest challenges for the present project.

Another example is platelet-derived growth factor-BB (PDGF-BB). PDGF is a mitogenic and angiogenic protein which can promote fibroblast growth. PDGF is a dimer consisting of two disulfide-bonded peptide chains, and the homo dimer PDGF-BB is the one with highest activity in promoting wound repair. New bone formation was significantly enhanced by PDGF when adsorbed on hydroxyapatite micro crystals. PDGF-BB is unique among several growth factors in enhancing both granulation tissue volume and the degree of re-epithelialization, stimulating granulation tissue formation in both normal and diabetic rats. PDGF delivered in collagen gel to treat tibial oeteotomies in rabbits enhanced functional fracture repair and stimulate osteogenesis significantly. PDGF delivered with the osteoporosis drug alendronate was also reported to substantially increase bone density.

Another embodiment is related to co-delivery of more than one agent. One example of this embodiment is the co-delivery of tobramycin and BMP-2.

Further details and representative examples of the present invention are described in the examples, which arc presented to show embodiments of the present invention and are not to be construed as being limiting thereof.

The scaffolds of the present invention, including the two-component polyurethane scaffolds exemplified herein demonstrate promise as tissue engineered scaffolds because they provide both porous structural supports for cell migration and new tissue formation, as well as local delivery of antibiotics to treat and prevent fracture-related osteomyelitis. Starting from a reactive liquid mixture, they potentially can be injected to cure in situ by a gas foaming process, allowing them to expand and fill irregularly shaped wounds. They have been shown to biodegrade to non-cytotoxic degradation products and facilitate cell proliferation and new tissue formation, both in vitro and in vivo. As shown here and in previous work, the dynamic mechanical properties and hydrophilicity can be adjusted by varying the level of poly(ethylene glycol). These effects primarily seem to result from glass transition temperature changes, causing the mechanical properties of the scaffolds to vary from glassy to elastomeric, although all materials demonstrate low permanent deformation and high resilience. Furthermore, as shown in this study, the presence of additives such as tobramycin can enhance the compressive strength, moduli, and elasticity of the scaffolds.

These PUR scaffolds exhibit tobramycin release, comparable to the release kinetics reported for PMMA and calcium sulfate bone cements. In embodiments of the present invention, there is a burst release of about 45%, 90%, and 95% with 0, 30, and 50% PEG, respectively, followed by a sustained release for approximately two weeks. The overall release of tobramycin is greater than that from PMMA cement beads, which are currently an established clinical therapy for elimination of osteomyelitis. These are clinically effective, but they exhibit low release efficiency and must be removed during a second surgery because they are not biodegradable. Furthermore, PMMA can be conducive to biofilm-forming bacteria, can reach unfavorably high temperatures during polymerization, and unreacted monomer can be cytotoxic. Biodegradable calcium sulfate pellets with 10 wt % tobramycin, approved for clinical use in countries outside the United States, have also successfully treated intramedullary infections and facilitated new bone growth in both animals and humans. These pellets offer a tobramycin burst release of 58%, with little more after 2-3 days; a more sustained release might be desired to avoid antibiotic resistance developed from subtherapeutic antibiotic levels. There also have been drainage and seroma formation issues associated with this material.

Alternatively, biodegradable PLGA microspheres provide sustained tobramycin release over one month, with relatively high encapsulation efficiencies of 40-60%. Control of the release profile can be achieved by varying the microsphere ratio of PLGA and PEG. Microspheres with 4.5-wt % tobramycin were implanted into a rabbit radial defect model infected with S. aureus, and after 4 weeks, the infection was eliminated and bone healing was observed. While these PLGA microspheres have been shown to be efficient antibiotic delivery vehicles, they must be pre-made, which precludes customization at the time of implantation or injection, and they do not possess the structural integrity typically associated with a scaffold.

Tobramycin release from the PUR scaffolds, as well as from the other referenced materials, is conjectured to be diffusion-controlled. Thus, as shown previously, release is independent of—and occurs on a shorter time scale than—the polyurethane degradation. When immersed in buffer (or serum), the scaffold swells with water, which dissolves any accessible tobramycin, allowing it to diffuse out of the scaffold into the surrounding media. The burst release may result from the immediate dissolution of any tobramycin located on or near the scaffold surfaces, with extended release resulting from eventual dissolution and diffusion of tobramycin embedded within the pore walls. The presence of PEG enhances the hydrophilicity of the otherwise hydrophobic polyester-based polyurethane scaffold, which increases the degree of swelling and rate of drug diffusion from the scaffold. The burst and overall rate of release also directly depend on the drug solubility, as observed experimentally. Drugs with lower water solubility than tobramycin tend to exhibit a lower burst release and more linear, longer-term release profiles. PEG may be desirable only in PUR scaffolds to enhance the delivery of such hydrophobic compounds.

The five primary amino groups in tobramycin potentially could be very reactive with the polyurethane, which reacts with free amines and hydroxyl groups during synthesis (the material is no longer reactive after synthesis is complete). Thus the tobramycin, as well as any added drug or growth factor, is added as a lyophilized powder to the hardener component of the polyurethane to limit reactivity. More tobramycin can be included in powder form than in liquid form, which could be limited by the solubility level within the very small volume of water added, and enables 100% encapsulation efficiency of tobramycin within the scaffold. This approach differs from a previously published method of incorporating ascorbic acid, which can stimulate osteoblast differentiation, in the polymer by reaction in the liquid phase with a prepolymer of lysine diisocyanate (LDI) and glycerol. The ascorbic acid was dissolved in glycerol prior to the reaction and, due to its four hydroxyl groups, reacted with the LDI to form urethane linkages and covalently bind to the polymer. Ascorbic acid release from the gas-foamed scaffold consequently was coupled to the material degradation rate.

The bioactivity of tobramycin released from the PUR scaffolds of the present invention was verified with Kirby-Bauer assays, suggesting negligible reaction between the lyophilized tobramycin and polyurethane during synthesis. Furthermore, due to the heat stability of aminoglycosides, activity seems to be unaffected by the slightly exothermic (up to 40° C.) polyurethane reaction. The Kirby-Bauer assays show that these scaffolds release sufficient tobramycin to exceed the minimum inhibitory concentration (4-8 μg/mL) and minimum bactericidal concentration (16 μg/mL) for S. aureus, while the time-kill assays suggest that these levels are sustained over the extended release time. The Kirby-Bauer results are consistent with the release curves, in that we observe higher inhibition from scaffolds with PEG than without, since the PEG scaffolds release a higher percentage of the incorporated tobramycin. Conversely, PMMA cement beads produce smaller ZI, as they exhibit significantly lower tobramycin release. PUR scaffolds containing colistin and tigecycline achieved similar Kirby-Bauer results (data not shown), demonstrating that this system can be used with other antibiotics besides tobramycin. The release and Kirby-Bauer assays were repeated with identical PUR scaffolds that had been sterilized by ethylene oxide treatment, to verify that future in vivo experiments would not be affected by the sterilization method. No differences were detected, both for tobramycin release rates as well as bioactivity of the tobramycin releasate.

The mechanical and biological properties of these PUR scaffolds can be adjusted to benefit a variety of wounds and applications. We have shown previously their potential utility in dermal wounds, such as bums and diabetic lesions. That study included the local delivery of platelet-derived growth factor, suggesting that the scaffolds can also carry growth factors or small molecule therapeutics to enhance healing. Similar materials were successfully used for cardiac regeneration, so the PUR scaffolds may benefit soft tissue wounds as well. While controlled release of biologicals has been shown before from polyurethanes, this seems to be the first such release from a reactive, injectable polyurethane scaffold.

Injectable, biodegradable polyurethane scaffolds provide both structural templates and antibiotic delivery vehicles for enhanced healing of infected fractures. Local tobramycin release from these reactive scaffolds potentially achieves higher local concentrations with lower systemic levels. The release profiles, characterized by a burst within the first 2 days followed by extended release for 30 days, can be tuned by the relative amount of PEG included in the scaffolds. While PEG was found to increase the cumulative release of tobramycin, it also substantially increased the burst release, thus incorporation of PEG may only be desirable in applications that require a higher burst of hydrophobic compounds. The tobramycin remains biologically active after sustained release. The versatility of this system enhances its potential for other uses, either with other antibiotics or for healing of tissues other than bone, such as infected soft tissue or dermal wounds.

The following examples are presented for exemplary purposes, and are not to be viewed as limiting the present invention.

### EXAMPLES

### Example 1

This Example demonstrates an aspect of the present invention, and more specifically a method of making a PUR scaffold of the present invention.

Glycolide and D,L-lactide were obtained from Polysciences (Warrington, Pa.), tertiary amine catalyst (TEGOAMIN33) from Goldschmidt (Hopewell, Va.), polyethylene glycol (PEG, MW 600 Da) from Alfa Aesar (Ward Hill, Mass.), and glucose from Acros Organics (Morris Plains, N.J.). Lysine triisocyanate (LTI) from Kyowa Hakko USA (New York), and hexamethylene diisocyanate trimer (HDIt, Desmodur N3300A) from Bayer Material Science (Pittsburgh, Pa.). PDGF-BB was obtained from Amgen (Thousand Oaks, Calif.). Sodium iodide (Na¹²⁵I) for radiolabeling was purchased from New England Nuclear (part of Perkin Elmer, Waltham, Mass.). Reagents for cell culture from HyClone (Logan, Utah). All other reagents were from Sigma-Aldrich (St. Louis, Mo.). Prior to use, glycerol and PEG were dried at 10 mm Hg for 3 hours at 80° C., and ε-caprolactone was dried over anhydrous magnesium sulfate, while all other materials were used as received.

Trifunctional polyester polyols of 900-Da and 1800-Da molecular weight (abbreviated as 900 and 1800) were prepared from a glycerol starter and 60% ε-caprolactone, 30% glycolide, and 10% D,L-lactide monomers, and stannous octoate catalyst, as published previously. These components were mixed in a 100-ml reaction flask with mechanical stirring under argon for 36 hours at 140° C. They were then dried under vacuum at 80° C. for 14 h.

PUR scaffolds were synthesized by one-shot reactive liquid molding of hexamethylene diisocyanate trimer (HDIt; Desmodur N3300A) or lysine triisocyanate (LTI) and hardener comprising either the 900-Da or 1800-Da polyol, 1.5 parts per hundred parts polyol (pphp) water, 4.5 pphp (1.5 pphp for LTI foams) TEGOAMIN33 tertiary amine catalyst, 1.5 pphp sulfated castor oil stabilizer, and 4.0 pphp calcium stearate pore opener. The isocyanate was added to the hardener and mixed for 15 seconds in a Hauschild SpeedMixer™ DAC 150 FVZ-K vortex mixer (FlackTek, Inc., Landrum, S.C.). This reactive liquid mixture then rose freely for 10-20 minutes. The targeted index (the ratio of NCO to OH equivalents times 100) was 115. To examine the effects of a hydrophilic polyether segment on the material properties, some materials were synthesized with poly(ethylene glycol) (PEG, 600 Da), such that the total polyol content consisted of 30 or 50 mol-% PEG and 70 or 50 mol-% of the polyester polyol.

Compression set of the scaffolds was determined using a TA Instruments Q800 Dynamic Mechanical Analyzer (DMA) in static compression mode (New Castle, Del.). After measuring their initial heights, triplicate 7 mm diameter cylindrical foam cores were compressed to 50% strain (i.e., 50% of their initial height) for 24 hours at room temperature according to ASTM standards. The samples recovered for 30 minutes, and then their final heights were measured. Compression set was calculated as the permanent deformation after the period of compressive stress, expressed as a percentage of the original height.

Core densities were determined from mass and volume measurements of triplicate cylindrical foam cores, of 7 mm diameter×10 mm height samples. The core porosities (ε_{C}) were subsequently calculated from the measured density values (ρ_{c}), where ρₚ=1200 kg m⁻³ is the polyurethane specific gravity and ρ_{A}=1.29 kg m⁻³ is the specific gravity of air.

$${ɛ}_{C} = 1 - ( \frac{{ρ}_{C}}{{ρ}_{P}} ) \frac{{ρ}_{P} - {ρ}_{A} {ρ}_{P} / {ρ}_{C}}{{ρ}_{P} - {ρ}_{A}}$$

The pore size and distribution were also assessed by scanning electron microscopy (Hitachi S-4200 SEM, Finchampstead, UK).

Temperature profiles of the reactive mixture during foaming were assessed with a digital thermocouple at the centers of the rising foams. Scaffold degradation rates in vitro were evaluated by measuring the mass loss at various time points up to 36 weeks of incubation of triplicate 10-mg samples in 1 ml phosphate buffered saline (PBS) (pH 7.4) at 37° C. as described previously. At each time point, the samples were rinsed in deionized water, dried under vacuum for 48 hours at room temperature, and weighed. The degradation media from 4 and 8 weeks were reserved for in vitro cell viability experiments.

### Example 2

This example describes how to make an example of the foam of the preset invention, and further describes tobramycin release.

A polyurethane foam of the present invention may be synthesized by two-component reactive liquid mixing of hexamethylene diisocyanate trimer (Desmodur N3300A) and hardener consisting of a poly(ε-caprolactone-co-glycolide-co-lactide) triol, poly(ethylene glycol) (PEG, MW 600), water, triethylenediamine catalyst, sulfated castor oil stabilizer, and calcium stearate pore opener using previously reported techniques. Lyophilized, powdered antibiotic (tobramycin or colistin) and glucose excipient were mixed thoroughly with the hardener component before foam synthesis, with a total solids maximum of 8 wt-%. Tobramycin-containing PLGA microparticles were likewise included at 25 wt-% in some of the foams.

In vitro release of tobramycin was measured from triplicate 20-mg foam samples each in 1 mL PBS at 37° C. 500 uL of the PBS was removed and refreshed at several time points from 0.5 to 28 days. The released tobramycin was quantified using a CBQCA Protein Quantitation assay. The activity of antibiotics released from the foam was evaluated by a standard Kirby-Bauer test. In these experiments, 6×2 mm foam discs containing tobramycin were placed on agar plates streaked with methicillin-susceptible S. aureus, while foams with colistin were plated on multi-drug resistant A. baumannii. The zones of inhibition were measured in comparison with standard 10-ug tobramycin discs after 24 hours.

The in vivo behavior of the foams was evaluated for biocompatibility, biodegradation, cellular infiltration, and tissue regeneration. 8×2 mm foam discs were implanted into full-thickness excisional dermal wounds in adult Sprague-Dawley rats. The wounds were splinted with stainless steel washers for 7 days to prevent wound contraction and thereby allow the normal wound filling and granulation tissue infiltration typical in humans. Semi-occlusive dressing held the foams in place and protected the wound. Wounds were harvested at days 5, 14, and 21 and processed for Gomori's trichrome histological evaluation. The osteoconductivity of the foams is currently being evaluated in a rat tibia fracture model.

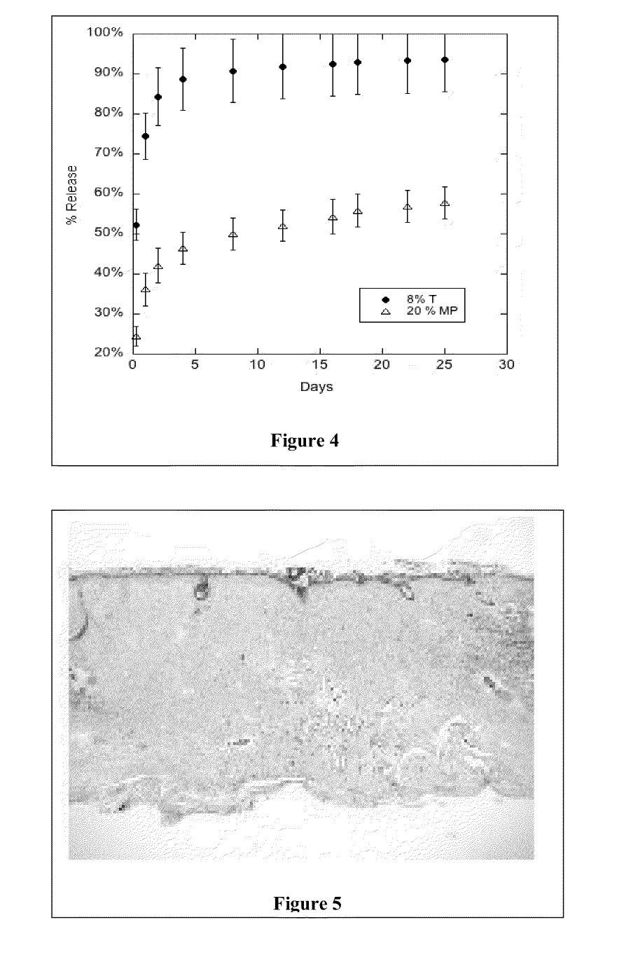

60-70% of the labile tobramycin eluted in PBS after 24 hours, with 90% released by 5 days (FIG. 4). The foams containing microparticles yielded a slower release, with 20-30% after 24 hours. The release curve was still increasing after 26 days of incubation, suggesting a continual, extended release of tobramycin from the microparticles.

The Kirby-Bauer tests revealed that sufficient levels of tobramycin and colistin diffused from the foams, and that this released antibiotic was effective against S. aureus and A. baumannii, respectively (Table 1, below). All zones of inhibition were larger than the minimum sensitivity levels of 15 mm. There was some variability due to slight differences in the sizes of the foam discs. As expected, foams with higher tobramycin content produced larger zones of inhibition. The inhibition zones from the microparticle foams were similar to those of the 2% tobramycin foams.

TABLE 1
Kirby-Bauer zones of inhibition (ZI) for tobramycin (T), tobramycin-
microparticles (T-mp), and colistin (C)-containing foam discs.

| Foam | ZI (mm) |
|---|---|
| 8% T | 34.3 ± 1.2 |
| 4% T + 4% G | 32.5 ± 0.9 |
| 2% T + 6% G | 26.2 ± 2.8 |
| 25% T-mp | 26.5 ± 2.5 |
| 8% C | 34.3 ± 1.3 |
| Control | 0 |

The foam showed extensive biodegradation in the excisional dermal wounds by day 21. Histology depicted plentiful cellular infiltration, as well as the presence of mature granulation tissue and dense collagen fibers, and neodermis formation for almost complete epithelialization. The wounds experienced little inflammation, with a transient giant cell response confined to the material remnants. The histological sections from the rat tibia fracture study are currently undergoing analysis. Upon preliminary evaluation, new bone tissue was well integrated with the scaffold, and there was no sign an adverse immune response.

### Example 3

This example demonstrates an additional method of making a foam of the present invention, including the incorporation of tobramycin.

Glycolide and D,L-lactide were obtained from Polysciences (Warrington, Pa.), tertiary amine catalyst (TEGOAMIN33) from Goldschmidt (Hopewell, Va.), polyethylene glycol (PEG, MW 600 Da) from Alfa Aesar (Ward Hill, Mass.), and glucose from Acros Organics (Morris Plains, N.J.). Tobramycin was obtained from X-Gen Pharmaceuticals (Big Flats, N.Y.), and hexamethylene diisocyanate trimer (Desmodur N3300A) was obtained from Bayer Material Science (Pittsburgh, Pa.). All other reagents were purchased from Sigma-Aldrich (St. Louis, Mo.). Prior to use, glycerol and PEG were dried at 10 mm Hg for 3 hours at 80° C., and e-caprolactone was dried over anhydrous magnesium sulfate, while all other materials were used as received. Simplex P cement beads with Tobramycin were obtained from Stryker (Mahwah, N.J.).

Polyurethane (PUR) scaffold synthesis. The 900-Da trifunctional polyester polyol was prepared from a glycerol starter and ε-caprolactone, glycolide, and D,L-lactide monomers at ratios of 60/30/10 (T6C3G1L) or 70/20/10 (T7C2G1L), and stannous octoate catalyst, as published previously. These components were mixed in a 100-mL three-neck flask with mechanical stirring under argon for 36 hours at 140° C. The polyols were then dried under vacuum at 80° C. for 14 hours. Two different polyol compositions were used to evaluate the effects of scaffold degradation times on the tobramycin release characteristics. The T7C2G1L polyol has a longer half-life (225 days) than T6C3G1L (20 days), causing the corresponding polyurethane scaffold to degrade more slowly.

The PUR scaffolds were synthesized by reactive liquid molding of the aliphatic hexamethylene diisocyanate trimer (HDIt) and hardener. The hardener contained the polyol, 1.5 parts per hundred parts polyol (pphp) water, 4.5 pphp TEGOAMIN33 tertiary amine catalyst, 1.5 pphp sulfated castor oil stabilizer, 4.0 pphp calcium stearate pore opener, and when appropriate, 20 pphp lyophilized tobramycin (8 wt-% of the final scaffold). The isocyanate was added to the hardener and mixed for 15 seconds in a Hauschild SpeedMixer™ DAC 150 FVZ-K vortex mixer (FlackTek, Inc., Landrum, S.C.), for a targeted index (NCO:OH×100) of 115. The resulting reactive liquid mixture then rose freely for 10-20 minutes. Some materials were synthesized with poly(ethylene glycol) (PEG, 600 Da), such that the total polyol content consisted of 50 or 70 mol-% polyester polyol with 50 or 30 mol-% PEG. The basic reaction scheme for the polyurethane network synthesis is illustrated below.

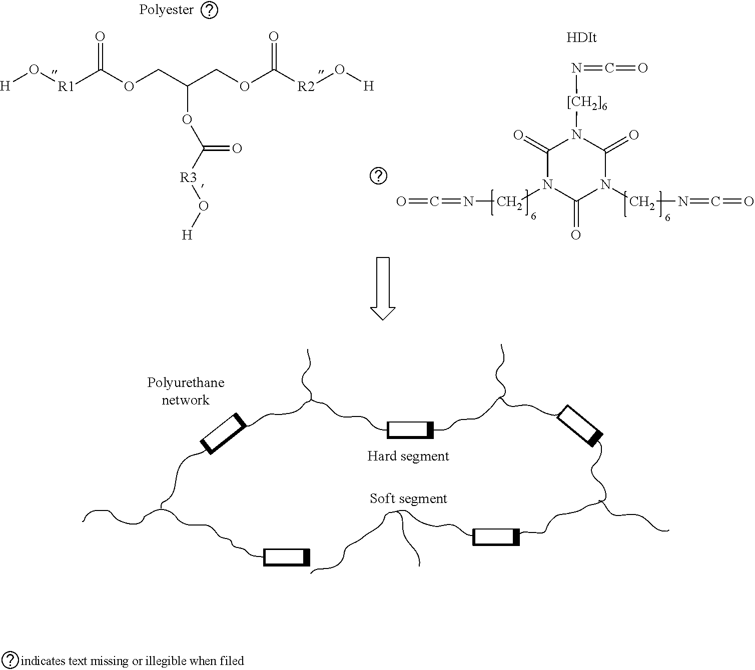

wherein R1, R2, and R3 are oligomers of e-caprolactone, glycolide, and D,L-lactide.

Core densities were determined from mass and volume measurements of triplicate cylindrical foam cores, of 7 mm diameter×10 mm height samples. The core porosities (cc) were calculated as shown herein. Pore size and distribution were also assessed by scanning electron microscopy (Hitachi S-4200 SEM, Finchampstead, UK).

Tobramycin was added as a powder to the hardener prior to reaction with the triisocyanate resin in order to minimize its reactivity with the reactive two-component polyurethane. Tobramycin's five primary amino groups otherwise cause it to react rapidly with isocyanates when in solution. Tobramycin is insoluble in polyester polyol, the primary component in the hardener; consequently the tobramycin remains in the solid phase during the chemical reaction. A loading of 8 wt-% was chosen to approximate the level of tobramycin delivered from the equivalent volume of PMMA cement beads, but higher loading can be achieved if necessary.

PMMA bead synthesis. The PMMA cement beads were made according to the manufacturer's instructions. Briefly, the liquid monomer was added to the bone cement powder and hand mixed. The resulting paste was rolled into individual 50-mg beads, approximately 5 mm in diameter.

### Example 4

This Example demonstrates in vitro release and antibiotic biological efficacy of the embodiment of Example 3.

In vitro release. Triplicate 500-mg samples of the scaffolds each in 1 mL phosphate buffered saline (PBS, pH 7.4) were mixed end-over-end while incubating at 37° C. At designated time points from 0.5 to 30 days, the buffer was removed from each vial and replaced. All release samples were frozen until analysis at the end of the 30 days. The released tobramycin was derivatized with o-phthaldialdehyde (PHT) and quantified with a Waters Breeze HPLC and UV detector, using a previously published method. 250 μL of each sample was added to 100 μL PHT (100 mg/mL in methanol) and 150 μL isopropanol. This mixture was vortexed for 30 seconds and incubated at room temperature for 45 minutes before injection. The injected sample (50 μL) was separated in an XTerra reverse-phase guard column (C8 5-μm 3.9×20 mm) and XTerra reverse-phase column (C18 5-μm 4.6×250 mm) and analyzed at 333 nm. The mobile phases were as follows: (A) 0.1% acetic acid in water, and (B) 88.5% acetonitrile in water with 0.1% acetic acid. Both were filtered through a 0.2 μm filter and degassed under vacuum. The buffer ratio was 80/20 A/B (A/B) for the first 2 min, with a gradual gradient to 77/23 (A/B) from 2 to 6 min. The samples were calibrated by an external standard curve from 0.05 μg/mL to 30 μg/mL. With a 1.0-mL/min flow rate, the tobramycin peak eluted at approximately 6.5 minutes.

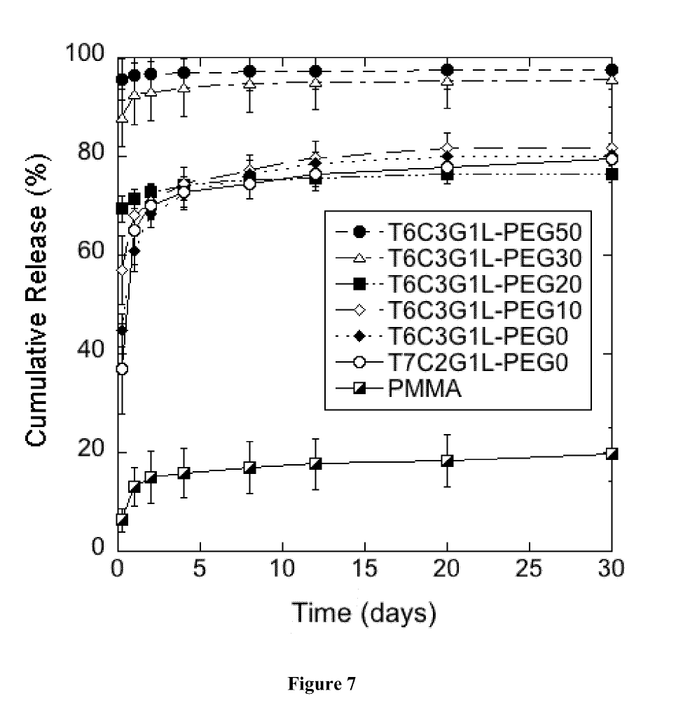

Tobramycin release profiles from the PUR scaffolds and PMMA beads are presented in FIG. 7. The scaffold degradation rate in vitro does not affect the release rate within this time scale, as the T6C3G1L-PEGO and T7C2G1L-PEGO scaffolds demonstrate similar tobramycin release profiles yet different degradation rates. The burst release increased from 45% to 95% as the PEG content in the polyol component was increased from 0 to 50%. Interestingly, there was a significant increase in the burst release of tobramycin when the PEG content was elevated from 20% to 30%. As the PEG content increased, the amount of tobramycin released at later time points (after the initial burst) decreased from 35% of the total release to <5%. Therefore, at the highest PEG content (50%), almost no additional antibiotic was released after the first 24 hours. After 30 days, the total release of tobramycin ranged from 70 to 95%, with 30 and 50% PEG scaffolds demonstrating the highest cumulative release. In contrast, the total release of tobramycin from the PMMA cement beads after 30 days was 20%, with little additional release after 7 days.

Antibiotic biological efficacy. The tobramycin activity was evaluated by Kirby-Bauer and time-kill experiments. For the Kirby-Bauer tests, colonies of methicillin-susceptible Staphylococcus aureus (S. aureus) from the American Type Culture Collection (ATCC 25923) were suspended in trypticase soy broth and the turbidity was matched to a 0.5 MacFarland standard. The bacteria were then streaked onto Mueller-Hinton agar plates (lower limit of detection was 20 CFU/ml). Tobramycin scaffold samples were cut into discs (6×2 mm, 400-600 μg tobramycin per disc) and placed on the agar plates. Zones of inhibition (ZI) were measured in comparison with 10-μg tobramycin BBL SensiDiscs (BD, Franklin Lakes, N.J.) and individual PMMA beads, with 3-4 mg tobramycin per bead, after incubation at 37° C. for 24 hours. The 10-mg tobramycin BBL SensiDiscs were chosen as a positive laboratory control, since this is a standard control used in pathology laboratories. Additionally, the bioactivity of the tobramycin after sustained release was evaluated. 0.5-μg tobramycin aliquots from release samples at 8, 20, and 30 days, as well as 0.5 μg pure tobramycin, were pipetted onto blank SensiDiscs. These discs were again placed onto S. aureus-streaked agar plates and the ZI were measured after 24 hours.

For the time-kill experiments, trypticase soy broth was inoculated with S. aureus and incubated for 18 hours at 37° C. Two dilutions of S. aureus were made in soy broth (10² and 10⁷ CFU/mL). Approximately 200 mg of foam containing tobramycin was added to each solution. 200-4 aliquots of broth were removed at the following time points: 0, 1, 2, 4, 12, 16, and 24 days and plated onto 5% sheep blood agar. S. aureus colonies were counted after incubation at 37° C. for 24 hours.

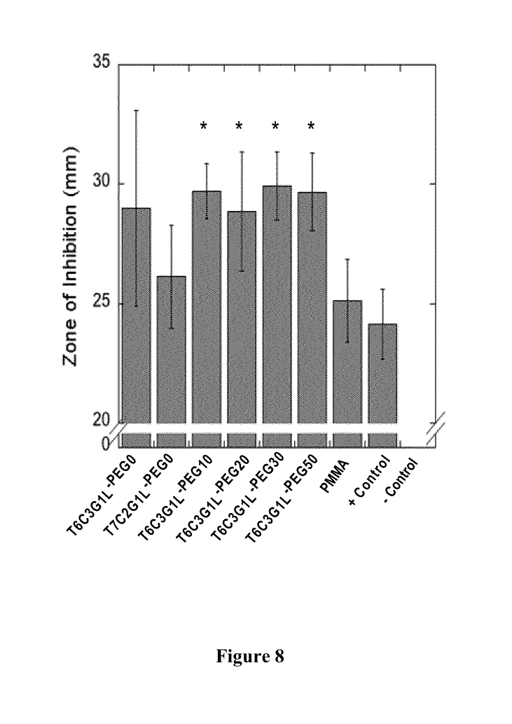

The bioactivity of the tobramycin released from the PUR scaffolds and PMMA cement beads over 24 hours was assessed by the standard Kirby-Bauer assay, as shown in FIG. 8. The zones of inhibition (ZI) generated by the PUR samples (400-600 μg tobramycin each) were consistently greater than both the positive laboratory control (10 μg each) and the PMMA beads (3-4 mg each). As might be expected from the release curves (FIG. 7), the PEG scaffolds produced larger ZI, with statistically significant differences from the positive control and PMMA beads (p<0.005). Furthermore, blank PUR scaffolds with no tobramycin generated no ZI. These data indicate that the tobramycin released from the PUR scaffolds in the first 24 hours is biologically active, as demonstrated by the observed inhibition of S. aureus growth.

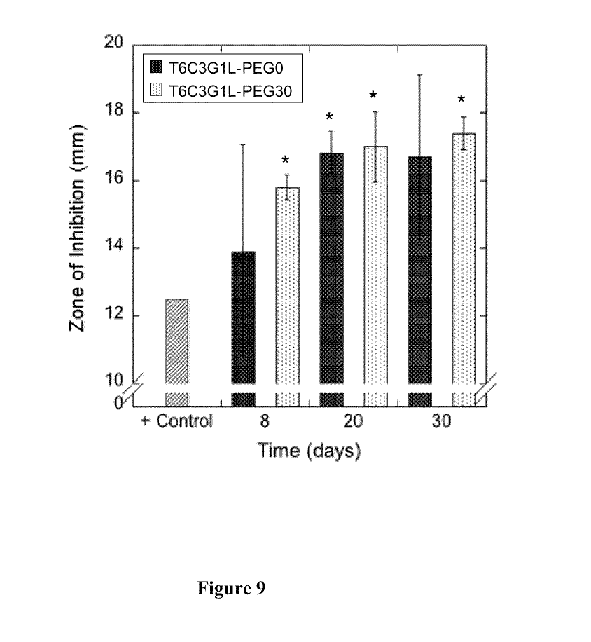

The bioactivity of the tobramycin released after 8, 20, and 30 days was analyzed in order to investigate the tobramycin stability and activity over time. The appropriate volumes of releasate containing 0.5 μg tobramycin per sample (as calculated from the release data in FIG. 3) were lyophilized, reconstituted in 10 μL PBS, and deposited on blank BBL SensiDiscs for Kirby Bauer tests. FIG. 9 shows the ZI for releasates from the T6C3G1L-PEGO and T6C3G1L-PEG30 scaffolds. These data indicate that the bioactivity of the tobramycin released from PUR scaffolds is comparable to that of the exogenous tobramycin control for up to 30 days. The time-kill experiments yielded zero bacterial colonies after each of the release time points, suggesting that the released tobramycin maintains its bactericidal activity for extended periods of time.

### Example 5

This example demonstrates mechanical properties and statistical analysis of the foam of Example 3.

Mechanical properties. Dynamic mechanical properties of a representative selection of scaffolds, both with and without tobramycin, were measured in compression mode. Cylindrical 7×6 mm samples were compressed along the axis of foam rise. The temperature-dependent storage modulus and glass transition temperature (T_{g}) of each material were evaluated under a temperature sweep of -80° C. to 100° C., at a compression frequency of 1 Hz, 20-pm amplitude, 0.3% strain, and 0.2-N static force. The stress relaxation modulus was evaluated as a function of time under 2% strain and 0.2-N static force. The frequency-dependent storage modulus was also evaluated by a frequency sweep of 0.1 to 10 Hz at a constant temperature of 37° C., with 0.3% strain and 0.2-N static force. Stress-strain curves were generated by controlled-force compression of the cylindrical foam cores at 37° C. With an initial force of 0.1 N, each sample was deformed at 0.1 N/min until it reached 50% strain (i.e. 50% of its initial height). The Young's (elastic) modulus was determined from the slope of the initial linear region of each stress-strain curve. The scaffolds could not be compressed to failure due to their elasticity, so the compressive stress was measured at 37° C. after one minute at 50% strain in the DMA stress relaxation mode, as a measure of compressive strength. Calculated from the measured force and cross-sectional sample area, the compressive stress indicates material compliance such that more compliant materials require lower stress to induce a particular strain.

Glass transition temperatures (T_{g}) of the PUR scaffolds were measured by DMA temperature sweeps in compression mode (Table 2). The T_{g} values ranged from 2.8-41.3° C. With exception of the non-PEG materials, tobramycin depressed the T_{g} with a variable effect on the scaffold mechanical properties. In previous studies, we observed a reduction in storage modulus at 37° C. coinciding with a decrease in T_{g}, but this trend seems to be confounded by the strengthening effect of tobramycin. The compressive stress (at 50% strain) and storage modulus at 37° C. consistently increased with addition of tobramycin, while the Young's modulus values showed no regular trend.

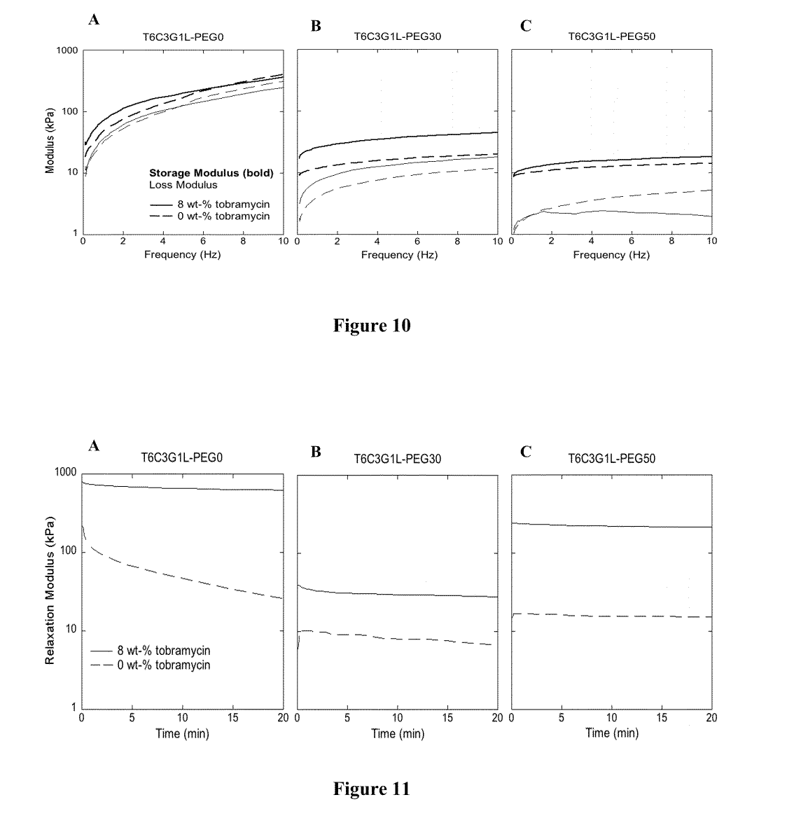

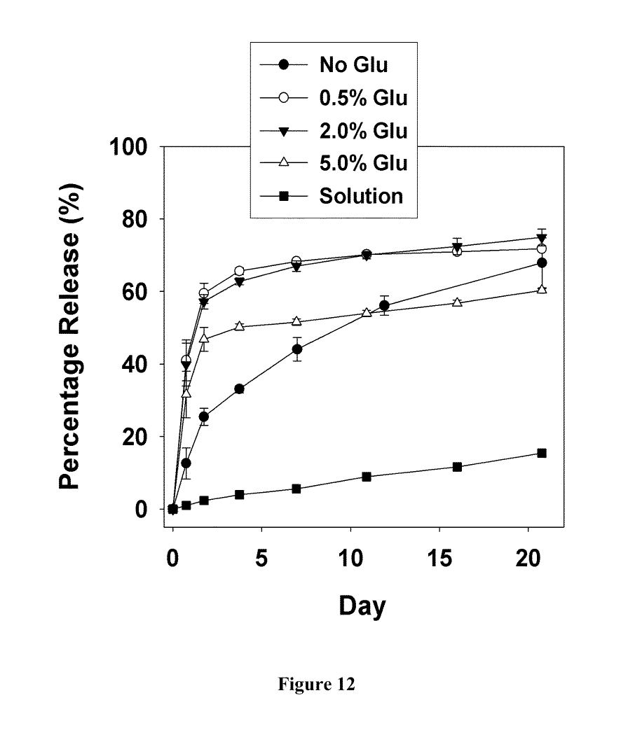

The frequency-dependent storage and loss moduli at 37° C. for some of the materials, both with and without tobramycin, are illustrated in FIG. 10. These representative materials were selected to illustrate the overall trends observed in response to the presence of PEG and/or tobramycin. The left panel depicts moduli of the T6C3G1L scaffolds without PEG. These materials have glass transition temperatures near 40° C., and their properties at 37° C. are representative of leathery materials in the glassy transition zone. The storage modulus (E′) and loss modulus (E″), which characterize the energy stored elastically and energy lost through viscous dissipation, respectively, were comparable and both rose by an order of magnitude with increasing frequency. As the PEG content of the materials is increased, the glass transition temperature is reduced to temperatures well below 37° C. The scaffolds therefore behaved more like ideal elastomers in the rubbery plateau zone, with moduli approximately an order of magnitude lower than the non-PEG materials. The storage modulus consistently remained well above the loss modulus, thus exhibiting less damping than the materials without PEG. The storage modulus was relatively constant over the frequency range, while the loss modulus increased by less than an order of magnitude. In all cases, the incorporation of tobramycin caused the storage and loss moduli, but not necessarily the Young's moduli, to be greater than those of otherwise equivalent scaffolds.

The stress relaxation curves displayed in FIG. 11 generally supported these findings. Tobramycin increased the relaxation moduli from 5- to 10-fold, while PEG caused them to decrease. The PEG scaffolds displayed elastomeric behavior, as the relaxation modulus increased initially with application of 2% strain but decreased almost negligibly over the following 20 minutes. The scaffold with neither PEG nor tobramycin was more leathery and exhibited a significant reduction in modulus over time due to rearrangement of the polymer network. Interestingly, tobramycin in this scaffold caused it to behave more like an elastomer, with little decrease in the relaxation modulus in response to the strain over time.Statistical Analysis. Statistical analysis of the results was performed using single factor analysis of variance (ANOVA). In cases where statistical significance is cited, the sample size is greater than or equal to three replicates per material.

PUR scaffold characterization. The density and porosity of the PUR scaffolds with and without tobramycin are shown in Table 2, below.

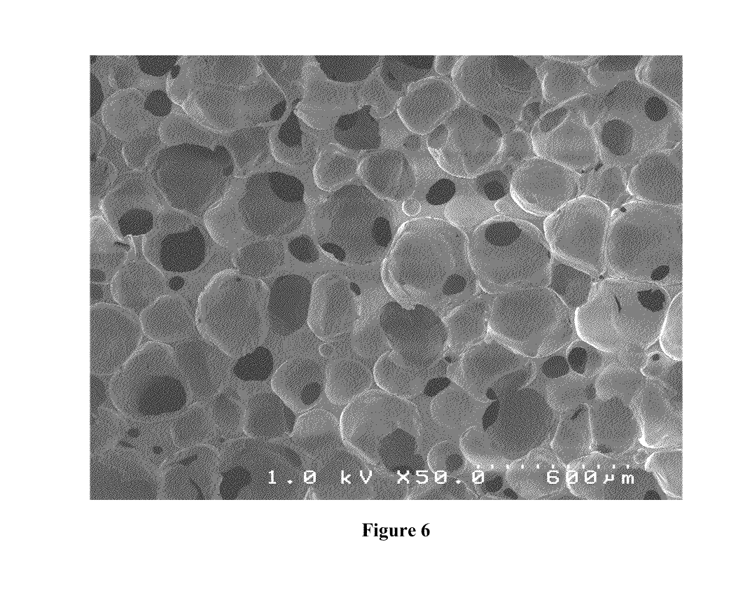

Incorporation of 8 wt-% tobramycin in the PUR scaffolds increased the density (and therefore decreased the porosity), although not with statistical significance (0.05>p>0.005). In most cases, the addition of PEG had an insignificant effect on PUR density and porosity. However, the T6C3G1L-PEG50 scaffold with tobramycin exhibited a significantly higher density than any of the other materials. SEM images of the PUR scaffolds are shown in FIG. 6.

### Example 6

This Example demonstrates advantages of aspects of the delivery features of the present invention.

### Local Delivery of PDGF-BB From PUR Scaffold Accelerates Tissue Regeneration In Rat Skin Excisional Wound Model

This example discusses two approaches that may be adopted to incorporate PDGF-BB into PUR scaffolds. The first approach was directly including PDGF-BB into the scaffold as a powder in the presence of excipients to enhance the release. For the second approach, PDGF-BB was bound to heparin-conjugated PLGA particles, coated with gelatin to form granules, and followed by incorporation the granules into the PUR scaffold. Both scaffolds had a porosity of more than 85%. The in vitro release of PDGF-BB from both strategies was similar, with a burst release for the first day followed by a sustained release for about one week. The released PDGF-BB promoted MC3T3 osteogenitor cell proliferation. The PUR/PDGF-BB implants at the size of 6 mm in diameter and 2 mm in height were fitted into rats' skin excisional wounds, and the presence of PDGF-BB within the scaffold attracts both fibroblast cells and microphage cells, promoting the scaffold degradation and regeneration of tissues.

### In Vitro Release of BSA-FITC From PUR/BSA-FITC Scaffolds

A scaffold of the present invention was prepared by one-shot reactive liquid molding of HDIt and hardener containing polyester triol and PEG. BSA-FITC was added to the hardener component prior to mixing with HDIt. Considering that proteins incorporate groups with active hydrogens (e.g., hydroxyl groups and amines), there is a concern that the protein will react with the polyisocyanate, resulting in damage to the protein. The data shown in FIG. 1 (12) suggest that a substantial portion of the BSA reacts with the polyisocyanate and is covalently bound to the scaffold, as evidenced by the low (<20%) release after 21 days. To protect the protein from reacting with the polyisocyanate, BSA-FITC was lyophilized with a varying amount of glucose excipient and then added to the hardener as a powder. As shown in FIG. 1 (12), the total amount of protein released is significantly higher when added as a powder compared to addition in solution. The glucose dosage plays an important role in the release profile; the presence of 0.5% and 2% glucose increased the total amount released after 21 days and increased the initial burst release. Further increasing the glucose dosage to 5% decreased the total release.

### In Vitro Release of PDGF-BB From PUR Scaffolds

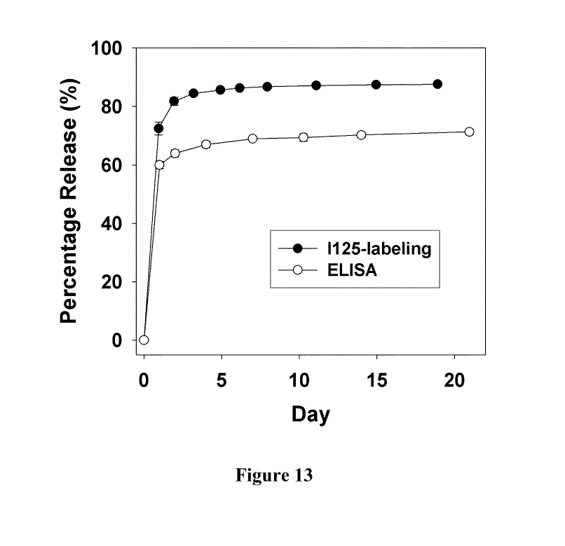

In contrast to the PUR/BSA-FITC scaffolds, addition of PDGF-BB as a powder without the glucose excipient results in negligible protein release, which is conjectured to result from the structural differences between BSA-FITC and PDGF-BB. To enhance the release of PDGF-BB, 2 wt % glucose was added to the scaffolds. The release profile was monitored by two methods. In the first approach, PDGF-BB was radiolabeled with iodine-125 (I-125) and a gamma reading machine was used to monitor the release kinetics. Release of PDGF-BB was also measured by ELISA assay using the liquid releasates. Both methods yield similar release profiles (FIG. 13), characterized by a burst release on the first day. The ELISA assay detected a lower total release (around 65%, compared with around 85% for I-125 labeling method), which is possibly due to denaturation of some fraction of the released protein which cannot be detected by its antibody.

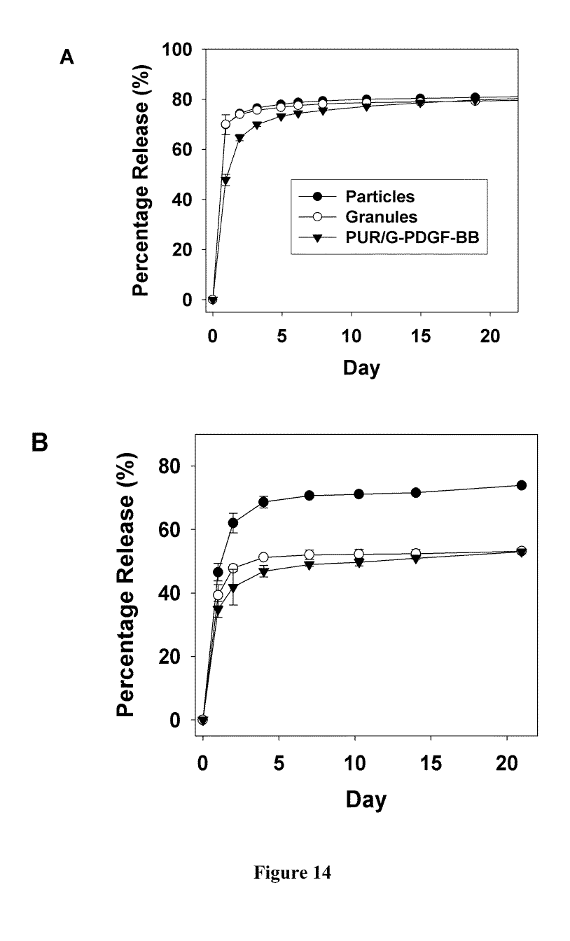

To mitigate the burst release, another strategy was adopted to achieve controlled release of PDGF-BB from polyurethane scaffolds. Heparin was bound to the surface of microparticles made from amine-terminated PLGA (average size 50 μm) using standard carbodiimide techniques. PDGF-BB was then loaded onto the particle surface through heparin binding. As shown in FIG. 14, the total release of PDGF-BB from the particles alone at day 21 was determined to be 80% and 72% by I-125 labeling and ELISA respectively. However, when the PLGA-Heparin-PDGF-BB microparticles were incorporated in the polyurethane scaffold, less than 10% of the total protein was observed to be released over 21 days (data not shown), which suggests that the surface-immobilized protein is reacting with the polyisocyanate. To enhance the stability of the protein on the PLGA particle surface, the particles were granulated by mixing the particles with a small amount of gelatin solution followed by forcing through a 48-mesh sieve to form 110-μm granules. The granules were then added to the hardener component before reacting with isocyanate to form PUR/G-PDGF-BB polyurethane scaffolds. The PDGF-BB release from the granules is similar to that of particles when detected by I-125 labeling (FIG. 14A), but lower when measured by ELISA (FIG. 14B). Compared with the release profiles from PUR/PDGF-BB scaffolds (FIGS. 13 and 14), the PUR/G-PDGF-BB scaffolds exhibit lower burst and more sustained release when measured by I-125 labeling. However, when measured by ELISA, the total release is lower for the PUR/G-PDGF-BB scaffolds, which again suggests that not all of the protein released is active in antigen-antibody interaction.

### In Vitro Cell Proliferation Assay of Released PDGF-BB

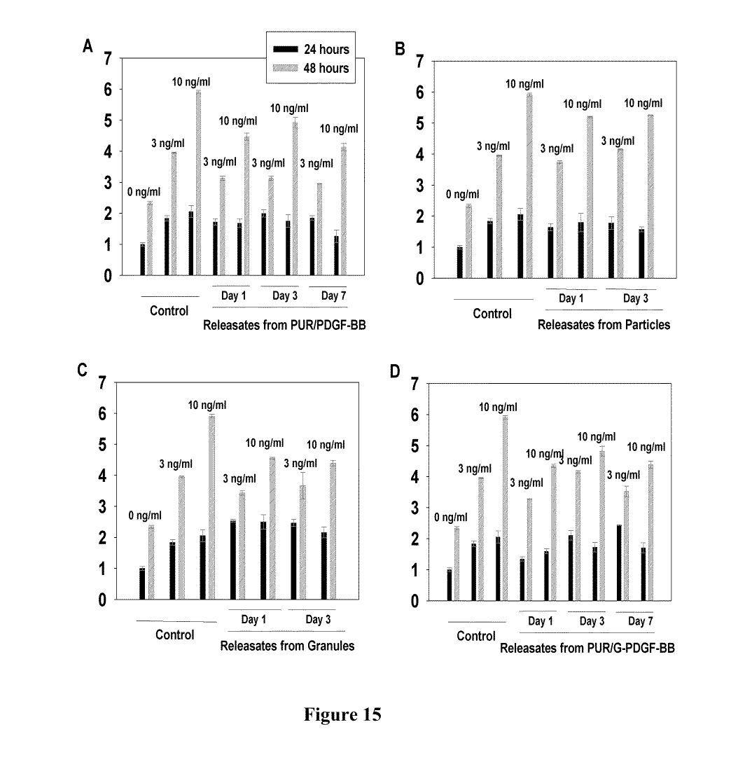

To assess the in vitro bioactivity of released PDGF-BB from the polyurethane scaffolds, releasates at day 1, 3, and 7 from PUR/PDGF-BB and PUR/G-PDGF-BB scaffolds were lyophilized and reconstituted to the same PDGF-BB concentration. After MC3T3 cells attached to the 96-well plates, the serum content in the cell culture medium was decreased from 10% to 2.5%. At the same time, the reconstituted releasates were added at dosages of 3 and 10 ng/ml. Fresh PDGF-BB (i.e., not released from the scaffolds but used directly as received from the supplier) was the positive control. Cell numbers were measured at time points of 24 and 72 h by staining the DNA and measuring fluorescence on a plate reader. FIG. 15 shows that the released PDGF-BB from both PUR/PDGF-BB and PUR/G-PDGF-BB are biologically active, promoting proliferation of MC3T3 cells in a time-dependent manner. At the 72 h time point, a dose-dependent effect was observed, although it was not evident at the 24 h time point. Compared with the positive control, the cell proliferation assay suggests that the released PDGF-BB exhibited 20% decreased activity.

### In Vivo Rat Skin Excisional Wound Test of PUR/PDGF-BB Implants

After the PDGF-BB release and bioactivity was identified in vitro, the PUR/PDGF-BB implants at the size of 6 mm in diameter and 2 mm in height were fitted into adult male Sprague-Dawley rats skin excisional wounds. The loading of PDGF-BB in the scaffold is 0 ug (control), 1.8 μg (low dose), and 18 μg (high dose) respectively. After 3 and 7 days post-implantation, the rats were sacrificed and the harvested implants processed with histology analysis. The presence of PDGF in the scaffold enhanced scaffold degradation, presumably by attracting microphage cells, as well as new granulation tissue formed by infiltration of fibroblast cells. As the healing progressed, new extracellular matrix with dense collagen fibers filled the defect. At day 7, a remarkable level of new tissue infiltration and scaffold degradation was observed for the PDGF samples, which is comparable to the effect of control scaffolds at day 21. Moreover, little inflammatory response or cytotoxicity was evident.

### Properties of PUR Scaffold

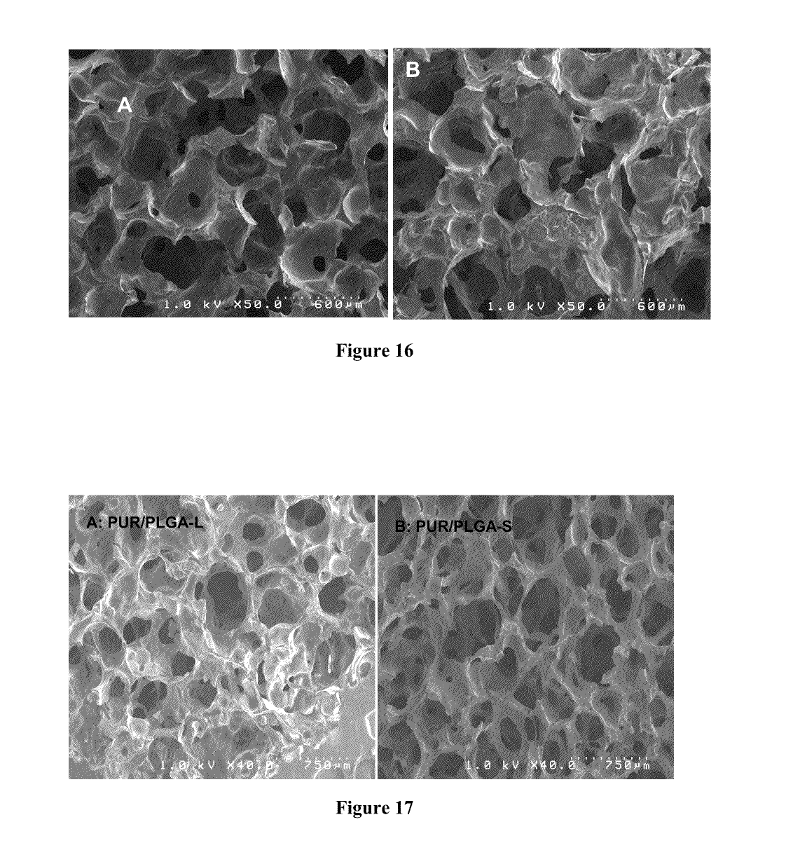

The polyurethane scaffold embodiments containing 2% glucose (FIG. 16A) is porous and the pores are interconnected as evidenced by SEM imaging. The sizes of the pores are in the range of several hundred microns. The presence of 15 wt % granules in the PUR scaffold (FIG. 16B) does not change the internal morphology very much. PUR/G has a somewhat lower density, thus its core porosity is somewhat higher than PUR. The porosities of PUR and PUR/G are calculated to be 87.41% and 85.22% respectively.

### BMP-2 Release From PUR Scaffolds And In Vitro Bioactivity

To achieve a long-term release and exert better bone wound healing effect, BMP-2 may be encapsulated into PLGA particles, followed by incorporation into PUR scaffold. To tune the release profile, three different particle sizes were chosen, with the average values of 80 μm (PLGA-L), 20 μm (PLGA-M), and 1 μtm (PLGA-S) respectively. The encapsulation of BMP-2 into large PLGA microspheres decreased the burst release of BMP-2 from PUR scaffolds. With decreasing size, the PLGA particles are more embedded in the scaffold, thus slowing the release and achieving a more sustained profile. While particles that are too small may not release BMP-2 at all because they are embedded so deeply in the scaffold, we conjecture that an optimal size range exists for the desired release profile. The released BMP-2 from PUR scaffolds is bioactive as verified by alkaline phosphatase (ALP) activity assay and Von Kossa mineralization assay performed on MC3T3 cells.

### Synthesis And Characterization of PUR Scaffold Containing BMP-2

PLGA microspheres were prepared using the double emulsion technique at different average sizes. PUR scaffolds were synthesized by one-shot reactive liquid molding of hexamethylene diisocyanate trimer (HDIt), and a hardener comprising 50 parts 900-Da polyol, 50 parts 600-Da PEG, and other essential compounds. BMP-2 powder in the presence of glucose (wt 2% in the foam) and heparin (20 fold that of protein), or PLGA microspheres was incorporated into PUR composite scaffolds through mixing with the hardener component before the foaming reaction.

The SEM image of PUR without PLGA particles is shown in FIG. 6A, and the inclusion of PLGA particles maintained the morphology property of PUR scaffold (FIG. 17). The polyurethane scaffolds contain interconnected pores with the size in the range of several hundred microns. This indicates that PUR scaffolds containing PLGA particles can serve as the matrix for cell growth and penetrating.

### Release of BSA-FITC From PUR And PUR/PLGA Scaffolds

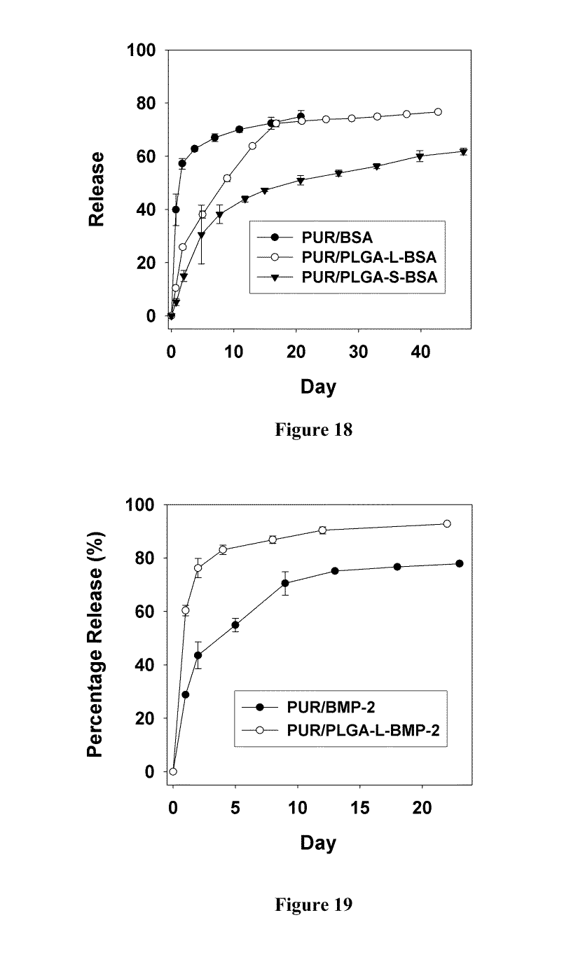

To evaluate the effect of encapsulation protein into PLGA particles on the release kinetics from PUR scaffold, small pieces of PUR scaffold incorporated with BSA-FITC powder or PLGA particles encapsulated with BSA-FITC were incubated in a-MEM containing 1% BSA under 37° C. the medium was changed as indicated in FIG. 18. The amount of BSA-FITC was determined by emission fluorescence at 530 nm after excitation at 485 nm. The BSA-FITC release profiles from PUR scaffolds shows a lower burst release by adopting the PLGA microsphere encapsulation strategy compared with directly incorporating BSA into PUR scaffold as powder; and a more sustainable release when decreasing the PLGA microsphere size from 80 μm to 1 μm (FIG. 18).

### Release of BMP-2 From PUR And PUR/PLGA Scaffolds

Small pieces of PUR scaffold incorporated with BMP-2 powder or PLGA particles encapsulated with BMP-2 were incubated in a-MEM containing 1% BSA under 37° C., and the medium was changed every 24 hours. Immunoassay (Human BMP-2 ELISA kit, from R&D systems) was adopted to determine the amount of BMP-2 released from the PUR scaffolds. Similarly, when BMP-2 was incorporated in PUR scaffolds as a powder, a burst release occurred on the first day and little BMP-2 was release after day 8 (FIG. 19). When BMP-2 as encapsulated into large PLGA microspheres at the average size of 80 μm and the particles embedded in PUR scaffolds, the burst release was substantially reduced and the release sustained up to 15 days (FIG. 19). Based on BSA-FITC release data, further decreasing the size of the PLGA particles is expected to achieve a more sustained profile, and the experiment is undergoing.

### In Vitro Bioactivity of Released BMP-2 From PUR Scaffolds

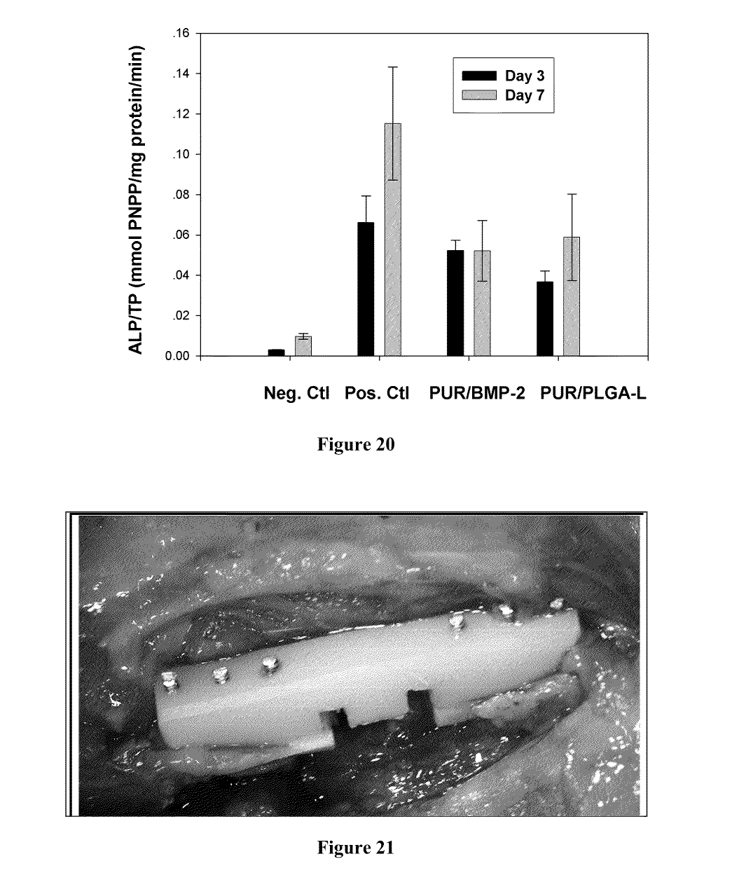

BMP-2 is known to stimulate alkaline phosphatase (ALP) expression and mineralization of MC3T3 cells. To evaluate the bioactivity of the released BMP-2, both ALP and mineralization assays (Von Kossa staining) were carried out. Based on the release profile, PUR/BMP-2 release samples from day 1 to 8 and PUR/PLGA-L-BMP-2 from day 1 to 10 were collected for the analysis respectively. Although weaker than the positive control, the BMP-2 released from PUR scaffold stimulated ALP expression of MC3T3 cells, which verified the in vitro bioactivity of the BMP-2 in the liquid releasates (FIG. 20).

### Example 7

This example helps demonstrate that biologically active molecules, including growth factors and antibiotics, can be released from biodegradable polyurethane scaffolds, and particularly that poly(ester urethane)urea/microparticle-tobramycin (PEUUR/MP-T) composite biomaterials promote healing of infected bone wounds in an established infection model

PUR foams were synthesized as shown herein.

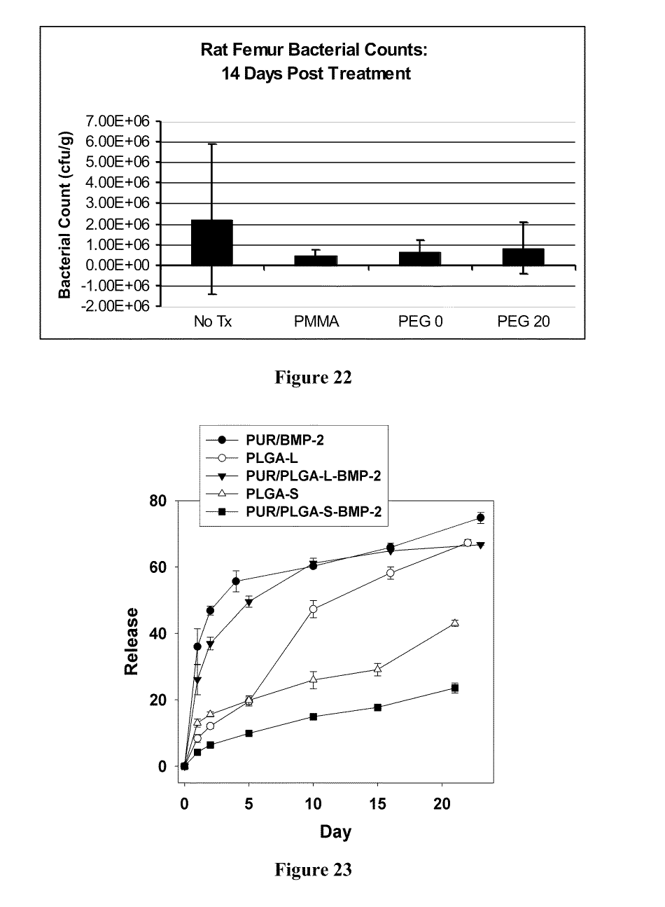

Evaluate the ability of PEUUR/MP-T composite biomaterials to promote healing of infected bone wounds in an established infection model. A pilot study in an infected rat segmental defect model was initiated on Apr. 7, 2008. The time point was selected as 2 weeks, and the T6C3G1L-PEGO and T6C3G1L-PEG30 materials were tested, along with a PMMA positive control. A photograph of the defect is shown in FIG. 21. The defect was infected by placing 30 mg of type I bovine collagen wetted with 100 μl Staphylococcus aureus Xen 36 in the wound (average Inoculating dose 3.30×105 cfu/ml) and debriding on day 14 post-infection. The test articles were then implanted into the defects and bacterial counts measured on day 14 post-implantation. Data are shown in FIG. 22.

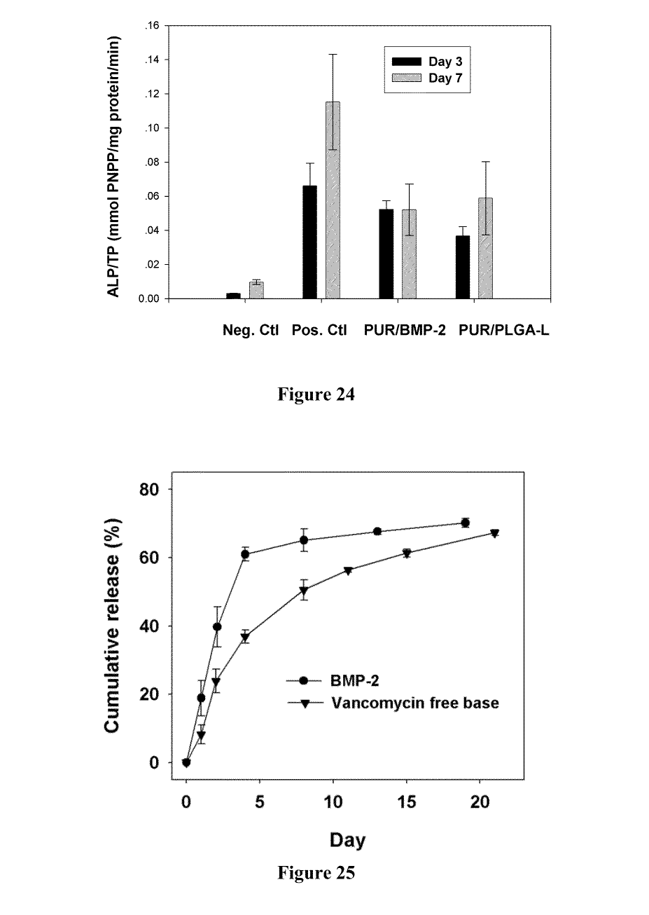

BMP-2 was reconstituted in PBS according to the manufacturer's instructions and mixed with heparin and glucose. The resulting solution was lyophilized to yield a dry powder, which was added to the hardener component of the PUR scaffold prior to mixing. Subsequently, Desmodur N3300A polyisocyanate (hexamethylene diioscyanate trimer) was added to the hardener component to prepare the PUR scaffold using published techniques. The PUR scaffolds each contained 2.5 μg BMP-2, 2 wt-% glucose (excipient), and 0.05 wt % heparin to stabilize the BMP-2. In some cases (PLGA-L and PLGA-S), BMP-2 was microencapsulated in PLGA (efficiency 80%) prior to incorporation in the polyurethane scaffold. In vitro release of BMP-2 in PBS at 37° C. was measured from 0 to 21 days by ELISA. The bioactivity of the released BMP-2 was determined by measuring alkaline phosphatase expression by MC3T3 cells incubated in released BMP-2 (FIG. 24). As shown in the Figure, the bioactivity of released BMP-2 is significantly greater than that of the negative control (no BMP-2) and less than that of the positive control (BMP-2 from the sample vial).

### Example 8

Methods: rhBMP-2 was purchased from R&D systems. V-FB was obtained by precipitation of vancomycin solution at pH 8. Porous PUR scaffolds incorporating BMP-2 and V-FB were then fabricated using a one-shot two-component reaction between the Lysine triisocyanate (LTI) and polyester triol as described previously (3). In vitro release experiment was carried out in PBS, with medium refreshed as indicated. The vancomycin concentration was determined by absorbance at 280 nm, and the BMP-2 concentration was measured by ELISA assay (R&D). The scaffolds were then tested in critical sized rat femoral segmental defects, contaminated or non-contaminated. After 4 or 8 weeks, the defects are harvested, and both the new bone formation and infection control were examined.

Results: Either BMP-2 or V-FB can be released from the composite PUR/BMP-2/V-FB, with profiles similar to reported when incorporating any of them individually in the PUR (FIG. 25).

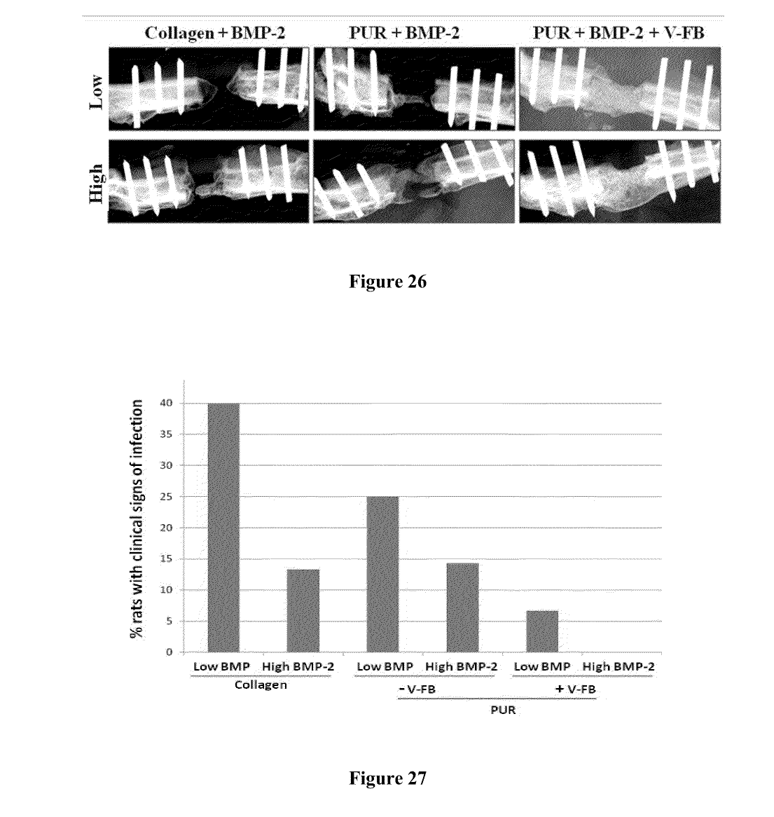

In order to test whether the presence of V-FB interfere with the osteogenic activity of BMP-2, the composites were first tested in the non-contaminated segmental defect for 4 weeks. Two BMP-2 dosages in PUR were chosen (Low: 2 ug per implant, and High: 20 ug per implant), with or without V-FB. The results showed that the presence of V-FB did not compromise the new bone formation promoted by BMP-2 (data not shown). Then the composites were tested in the contaminated segmental defect for 8 weeks, using collagen sponge soaped with BMP-2 as control. FIG. 26 showed that, at both low and high BMP-2 dosages, PUR performed better than collagen sponge due to more sustained release. Furthermore, in the presence of V-FB, the new bone formation was enhanced due to infection control by long-term release of antibiotic.

Interestingly, the trend of new bone formation is consistent with the clinical infection rate observed. As shown in FIG. 27, when counting the number of rats with infection at the end of the experiment, PUR treatment groups has less infection rate compared with collagen, and the infection decreases when V-FB is present. The PUR treatment group with both high BMP-2 dosage and V-FB showed no clinical signs of infection.

Conclusions: In the contaminated segmental defect, both infection control and new bone formation were achieved synergistically through dual release of V-FB and BMP-2 in a sustained manner. The outcome of this study suggests that the biodegradable PUR/BMP-2/V-FB composite may be a potential strategy for the clinical treatment of bone wounds complicated by infection.

### Example 9

This example shows that allograft/PUR composites of the present invention, even without addition of a growth factor such as rhBMP2, are both a safe and effective bone void filler.

The clinical need. During the course of OEF and OIF, there have been >8,000 open fractures, most of which occur to the tibia. Furthermore, ˜67% of the tibia fractures are the most challenging Type 3 open diaphyseal fractures. Open fractures are fraught with complications, particularly among the military population, presumably because of the nature of the wounds. A retrospective study has reported that 18 of 35 Type 3 open tibia fractures became infected. Ten of these 35 fractures resulted in delayed unions and 5 of the limbs were amputated, primarily due to infectious complications. These findings underscore the fact that the current standard of care for Type 3 fractures, particularly the bone graft, is inadequate.

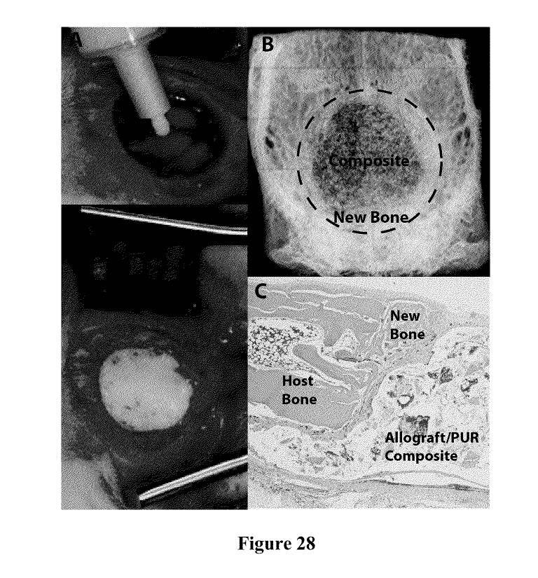

The proposed solution. To address this clinical need, we have developed an injectable composite scaffold comprising allograft bone particles and a lysine-derived polyurethane (PUR). These materials have tough mechanical properties, undergo controlled degradation to non-toxic decomposition products (e.g., lysine and a-hydroxy acids), and have tunable porosity. Furthermore, they are fabricated by reactive molding, thus rendering them suitable for injection or application as conformable pastes. Injection of the composite into a 15-mm rabbit calvarial defect is shown in FIG. 28. The working time is 3-5 min, during which time the material is mixed and injected into the wound, where it conforms to the shape of the defect and cures to form a solid porous scaffold in 10-12 min (FIG. 28(A)). The scaffold is rapidly infiltrated, resorbed, and replaced by new bone. The radiograph in FIG. 28(B) shows new bone formation extending ˜2-3 mm into this critical-size defect after 6 weeks, whereas the empty defect negative control generally exhibited <1 mm new bone formation (data not shown). One rabbit died on day 12 post-surgery due to complications unrelated to surgery, and the defect was examined histologically for signs of inflammation and tissue reaction (FIG. 28(C)). A modest amount of new bone formation was observed at the periphery of the defect, and no inflammation or other adverse tissue response was observed near the dura.

### Example 10

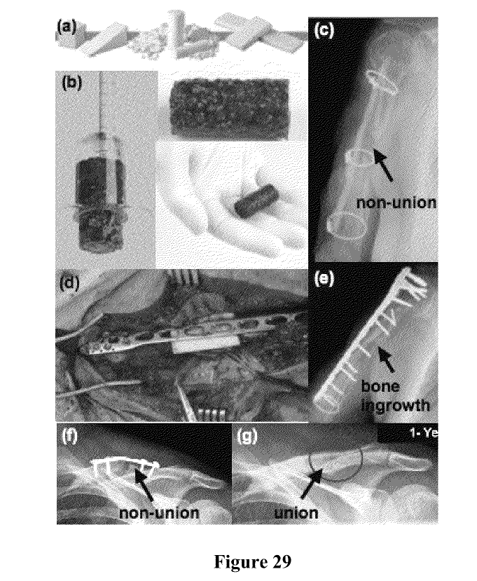

PLEXUR™ (available from Osteotech) allograft/polymer biocomposites promote healing when implanted in human patients. To overcome the limitation of slow remodeling, porous allograft bone/polymer composites that remodel by creeping substitution and cellular migration have been developed. In previous studies, the osteoconductive filler content ranged from 4-18 vol %. Due to its relatively low volume fraction, the filler was completely embedded in polymer, thus the rate of remodeling scaled with the rate of polymer degradation. Furthermore, the particle size of the mineralized filler was generally <20 μm, which is below the preferred size range for remodeling by creeping substitution. By incorporating >100 μm allograft bone particles and a modest (e.g., <50%) amount of porosity, composites that support rapid cellular infiltration and remodeling have been developed. The clinical success of this approach is demonstrated by Osteotech's Plexur™ technology platform based on allograft bone/polymer composites (FIG. 29), which are being developed as a continuous product pipeline. Osteotech has launched two of these products to the market, namely Plexur™ P and more recently Plexur™ M.

Both products are approved by FDA for use in orthopedic and spine applications, have been widely accepted by the surgical community, and have been used for hundreds of surgical cases to date. Plexur™ P is a highly porous biocomposite scaffold consisting of cortical allograft bone fibers and biodegradable poly(DL-lactide-co-glycolide) (PLGA). Interconnected pores facilitate rapid cellular infiltration and absorption of blood, thus providing an osteoconductive material that facilitates bone in-growth and integrates with host bone. Plexur P has enabled surgeons to treat bone defects which were previously difficult to treat. In the following case, pre-operation an older woman had a persistent non-union of the proximal humeral shaft. Plexur P inserted in the non-union site to stabilize the defect and to establish an osteoconductive pathway. The defect was fixed with a locking compression plate to provide stabilization. After only 3 months, bone can be observed growing into the defect site. The implant remodeled and the patient was able to move her arm voluntarily after 7 years. In a second case, a 32-year old male with an infected non-union of the clavicle underwent revision surgery where the wound was re-opened, the old hardware removed, and Plexur™ P implanted in the defect site secured by new plates. A post operative x-ray shows the positioning of Plexur™ P with a new stabilizing plate. Healing and union occurred at the defect site at 3 months, and 1-year post-operative follow up x-ray showed complete union with ingrowth of new bone grown scaffold degradation. The plates were removed and the defect area showed complete healing without any infection.

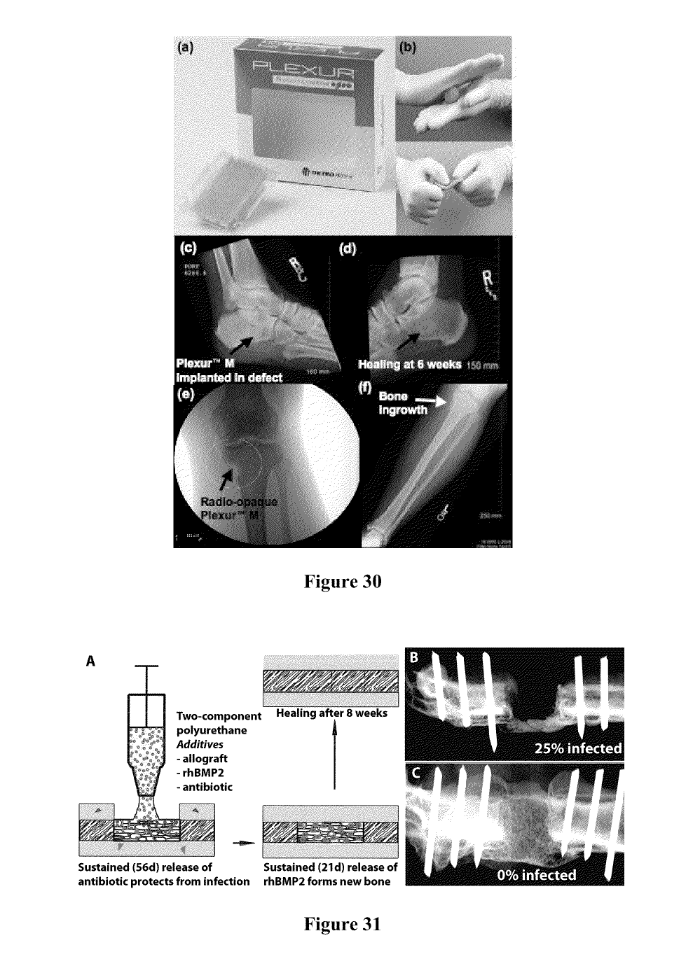

Plexur™ M is the most recent biocomposite product manufactured from allograft bone fibers and PLGA. As shown in FIG. 30(a)-(b), the composite is warmed to a moldable state and then implanted to fill a bony defect. At body temperature the composite cools, hardens, and sets. At this point, Plexur™ M can be machined, drilled and reamed to accommodate various instrumentations. Within the body, pores are formed throughout the implant through dissolution of a porogen, followed by cellular infiltration, remodeling, and integration with host bone. In one case, the patient had a calcaneous fracture which was curetted and burred to clean out the defect. Plexur™ M was heated and molded to conform to wound site, where it hardened in situ to fit the contour of the defect (FIG. 30(c)). Bone began to grow into the defect, and only after 6 weeks the defect began to fill up with host bone (FIG. 30(d)). In another surgical case of tumor osteoid osteoma, the proximal tibia was partially excised and Plexur™ M was implanted in the surgically debrided defect. Immediate post-operative pictures showed the presence of radiopaque Plexur M in place (FIG. 30(e)). The 7-week post-operative follow up x-ray of the defect shows that bone has grown through the Plexur M and the graft has almost completely remodeled and integrated into host bone.

Two-component polyurethanes support the development of an injectable allograft/polymer biocomposite. The next generation Plexur™ product is Plexur™ LV, an injectable biocomposite. While PLGA has an established history as a component in FDA-approved biomedical devices, it is a thermoplastic polymer and thus cannot be injected. Two-component polyurethanes (PUR) are useful for injectable applications due to the fact that they can be processed as a reactive liquid that subsequently cures in situ to form a solid porous elastomeric scaffold. Biodegradable PUR scaffolds synthesized from lysine-derived polyisocyanates and polyester polyols have been shown to degrade to non-toxic compounds and support cell attachment and proliferation in vitro. These materials also have tunable degradation rates, which are highly dependent on the choice of polyol and isocyanate components. Polyurethanes have tunable mechanical properties, which can also be enhanced with the addition of fillers, and exhibit elastomeric rather than brittle mechanical properties. The addition of osteoconductive fillers such as—TCP has been reported to increase not only the mechanical properties, but also the extent of bone ingrowth and new bone formation. Therefore, lysine-derived two-component polyurethanes are a safe and useful choice for the development of the injectable Plexur™ LV biocomposite. Future products in the development pipeline include a Plexur™ LV bone void filler for craniofacial applications, as well as Plexur™ LV weight-bearing composites, which are under development for repair of tibial plateau fractures and mandibular defects.

A polyurethane-based bone cement (Kryptonite™) has been approved by FDA. On Nov. 18, 2009 Doctors Research Group (DRG) received 510(k) approval to market kryptonite bone cement for cranioplasty applications. It is anticipated that Kryptonite™ will serve as a useful predicate device in Osteotech's regulatory filing, considering that it is a polyurethane-based device. This material comprises three components: an MDI prepolymer, a castor oil polyol, and calcium carbonate to yield an injectable paste. However, Kryptonite™ is manufactured from methylene diphenyl diisocyanate (MDI), which is a potential toxicity risk due to its possible degradation to methylene diphenyl diamine (MDA), an acutely toxic and carcinogenic aromatic diamine. Furthermore, due to the small size of the CaCO₃ particles, the overall rate of degradation and remodeling of Kryptonite™ compared to Plexur™ LV is anticipated to be slow, which has been shown to impede bone growth. In summary, the Plexur™ allograft/polymer composite technology is a clinically-proven approach for healing bony defects. As the next-generation product in the Plexur™ platform, Plexur™ LV will build on the commercial success of Plexur™ M and P by introducing a material with the clinically desirable feature of injectability.

Reactive two-component PUR scaffolds are an effective delivery system for antibiotics and growth factors. In the current treatment regimens, contaminated bone defects are debrided, systemic antibiotics are given, and the wound is treated with antibiotic-laden poly(methyl methacrylate) (PMMA) beads followed by bone grafting. Healing can be accelerated by adding growth factors to the bone graft; however, infection is known to inhibit new bone formation, even in the presence of an osteogenic factor. To address this clinical need, we proposed a new concept—a dual-delivery device. Although the systemic antibiotics and PMMA/antibiotic beads control the infection, it is often not completely eliminated. Thus, after grafting and definitive closure of the wound, the implanted bone graft, a foreign material in an avascular and often contaminated defect, functions as a nidus for infection. The present inventors discovered that dual delivery of an antibiotic and recombinant human bone morphogenetic protein-2 (rhBMP2) would protect the bone graft from infection, allowing healing to proceed unimpeded. We have accomplished this goal using an injectable polyurethane (PUR) delivery system. Prior to injection, the two liquid components of the PUR are mixed, at which point biologically active components, such as allograft bone particles, antibiotics, and/or rhBMP2, are added to the reactive mixture. Sustained release of rhBMP2 for up to 21 days has been achieved, as well as release of vancomycin (V) at concentrations exceeding the MIC for S. aureus for over 8 weeks. We believe that long periods of sustained release are important because it takes weeks to months for functional blood vessels to form in large defects such as the ones we are targeting. In a non-contaminated defect, the PUR dual-delivery device (PUR+V+rhBMP2) regenerated more bone than the current standard of care (rhBMP2 on a collagen sponge). In a more clinically relevant infected rat femoral segmental defect model, the novel PUR+V+rhMBP2 implants generated more new bone than the collagen+rhBMP-2 device and protected the scaffold and wound site from infection (FIG. 31).

Summary. An allograft/polyurethane biocomposite has been developed to address the clinical need for an injectable bone void filler that supports rapid cellular infiltration and remodeling. The biocomposite is undergoing commercial development as Plexur™ LV, which is the next-generation product in Osteotech's clinically-proven Plexur™ platform of biocomposites. Preclinical data show that Plexur™ LV is both a safe and effective bone void filler, and also supports the delivery of biologically active antibiotics and growth factors. These features, which differentiate Plexur™ LV from existing bone void fillers, are anticipated to enable surgeons to more effectively restore wounded Warriors with contaminated Type III open fractures to function.

### Example 11

Plexur™ LV will improve infected open fracture outcomes. To return injured Warriors to function, each complication and barrier to restoring the limb must be addressed. Infection and nonunion are common and result in significant morbidity and loss of limb. In the current standard of care, contaminated bone defects are debrided, systemic antibiotics are given, and the wound is often treated with antibiotic-impregnated PMMA beads followed by bone grafting. Healing can be accelerated by adding growth factors to the bone graft; however, infection is known to inhibit new bone formation, even in the presence of an osteogenic factor. Thus due to both the large size of the defects that Warriors sustain, as well as the presence of infection, surgeons frequently combine an FDA-approved biomedical device with FDA-approved biologics to improve patient outcomes. Through OETRP funding, the PI and CoI Wenke have shown that dual delivery of vancomycin and rhBMP2 from PUR scaffolds protects the implant from infection and accelerates healing relative to the clinical standard of care (rhBMP2 delivered on a collagen sponge). Our solution is a simple, off-the-shelf device that can be customized to treat patients at the point of care. Its handling properties (e.g., working and setting times) are comparable to those of existing calcium phosphate cements, thus making it easy to use. However, the cured material has plastic (not brittle) mechanical properties and rapidly supports cellular infiltration through the processes of cellular migration and creeping substitution. Plexur™ LV will not be marketed by Osteotech for delivery of biologics. However, the PI, CoI Wenke, and other researchers at USAISR are investigating the utility of the product as a delivery system for rhBMP2 and vancomycin in pre-clinical models (AFIRM Projects 4.5.1 and 4.5.3). It is anticipated that these preclinical studies funded by OETRP and AFIRM will guide surgeons on how to combine Plexur™ LV with FDA-approved biologics. The advantages to the wounded Warriors will include both fewer required surgeries as well as accelerated healing, and potentially reduced frequency of infectious complications.

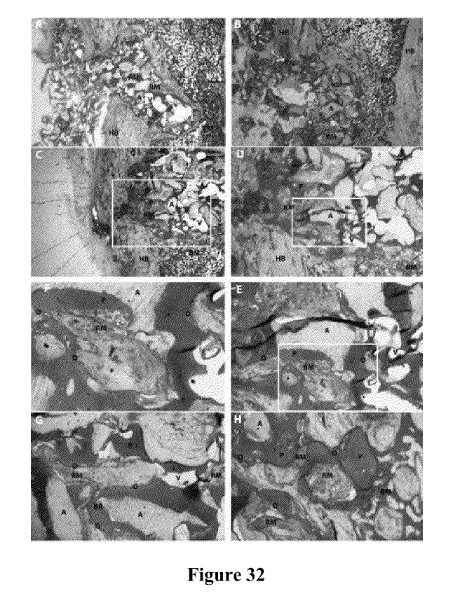

Injectable two-component allograft/PUR composite scaffolds support new bone formation. Porous allograft/PUR composites remodel by creeping substitution and cellular migration. In previous studies, the osteoconductive filler content ranged from 10-40 wt % (˜4-18 vol %). Due to its relatively low volume fraction, the filler was completely embedded in polymer; thus the rate of remodeling scaled with the rate of polymer degradation. Furthermore, the particle size of the mineralized filler was generally <20 μm, which is below the preferred size range for remodeling by creeping substitution. To address this limitation, we aimed to accelerate the rate of remodeling of bone/polymer composites by incorporating >100 μm allograft bone particles and a modest (e.g., 30-60%) amount of porosity. We reasoned that increasing the allograft content while maintaining porosity would accelerate cellular infiltration into the composites through both migration of cells into open pores, as well as remodeling of allograft particles by creeping substitution. Thus we investigated the effects of porosity on the mechanical and processing properties of bone/polyurethane composites comprising 45-50wt % (31-36 vol %) allograft bone particles, which was the highest allograft loading achieved for an injectable system. To evaluate the in vivo biocompatibility and remodeling of the allograft/polymer composites, the composition representing an optimum balance of porosity and initial mechanical properties was injected into bilateral femoral condyle plug defects in athymic rats. Thin (e.g., 4-6 m) decalcified sections stained with fuchsin red/toluidene blue mixture are shown in FIG. 32 after 3 weeks. FIG. 32(A) corresponds to the case where the material was injected and the wound immediately closed, while FIG. 32(B)-(C) correspond to the case where the wound was closed 15 minutes after injection. FIG. 32(D)-(F) are higher magnification views of the material shown in FIG. 32(C). Polymer is stained red, unresorbed allograft and cortical bone are stained light pink, nuclei are stained purple, and collagen and connective tissue are stained blue. Direct apposition of the polymer (labeled “P) against the host bone (labeled “HB”) surface is evident in the histological sections, suggesting that the injected composite established close contact with the host tissue. There is evidence of new bone growth adjacent to the material, as well as regions of active remodeling (labeled “RM”) near the host bone/composite interface and also deep into the interior of the composite. These regions of active remodeling exhibit evidence of allograft resorption, osteoid (O) formation, collagen deposition, and new bone formation. While there is extensive remodeling of allograft particles throughout the composites, some of the allograft particles (labeled “A”) were embedded in the polymer and thus did not remodel. Cells appeared to infiltrate the material both by entering open pores (voids labeled “V”), as well as via resorption of allograft particles, as shown in FIG. 32(G)-(H). FIG. 32(G) shows the cellular pathway in an interior region of the composite, while FIG. 32(H) shows the infiltration of cells near the composite/host bone interface, where cells from the marrow (labeled “BM”) are observed to migrate into the composite. In the center of FIG. 32(H) there is an allograft particle undergoing active remodeling that appears to be embedded in polymer except for a small breach (labeled “B”) where cells infiltrated along the allograft/polymer interface. Similarly, FIG. 32(E)-(F) show a large allograft particle that appears to be embedded in polymer except for two breaches where cells have begun to infiltrate along the allograft/polymer interface. These embodiments show that resorption of the allograft creates pores into which cells subsequently migrate, thereby presenting an alternative pathway (in addition to migration through open pores) by which cells can infiltrate the composite.

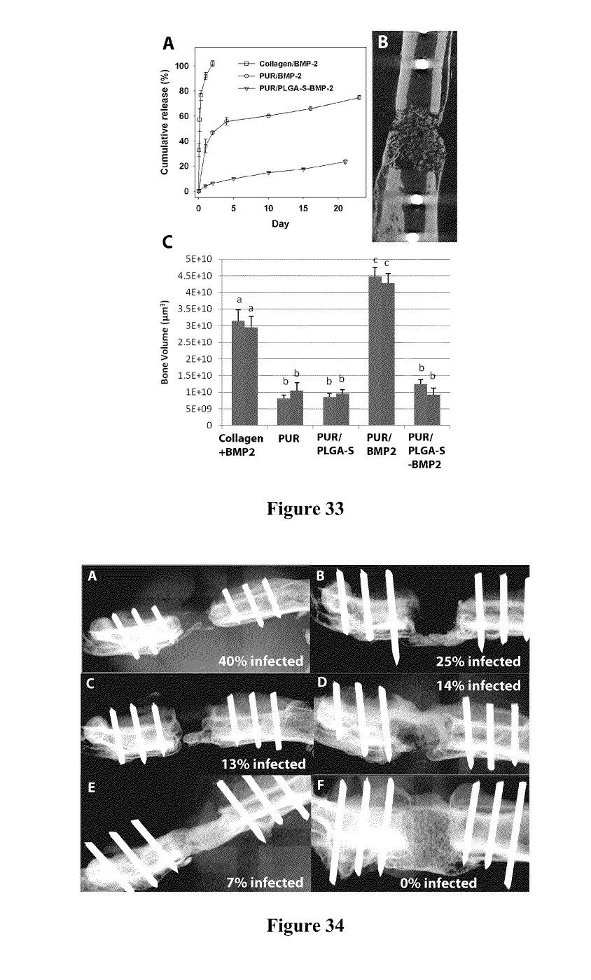

Growth factors released from two-component polyurethanes with tunable release kinetics enhance new bone formation. Another advantage of PUR scaffolds is that the two-component reactive two-component molding process enables customization using added biologics (e.g., growth factors and/or antibiotics) at the point of care. While previous studies suggest that the allograft/PUR composites are osteoconductive, treatment of combat-related orthopaedic traumatic injuries requires the use of more osteoinductive therapeutics. rhBMP2 delivered from a collagen sponge (INFUSE® Bone Graft, Medtronic) is an FDA-approved therapy for posterior-lateral spine fusion, tibial fractures, and specific craniofacial applications. The collagen sponge delivery system results in a bolus release of growth factor in the first 24-48 hours (FIG. 33(A)), but a number of studies have suggested that sustained release of rhBMP2 is more effective for promoting new bone formation. In order to modulate the release kinetics, rhBMP2 (60 μg/ml) was incorporated in PUR scaffolds by either direct addition of a labile powder or by encapsulation in large (L) or small (S) PLGA microspheres prior to incorporation in the scaffold. As shown in FIG. 33A, the labile powder (PUR/BMP2) formulation resulted in a burst followed by a sustained release of rhBMP2 up to day 21. Encapsulation of rhBMP2 in ˜1 μm PLGA (50/50 L/G, Mₙ˜50,000 g/mol) microspheres prior to incorporation in the PUR scaffolds essentially eliminated the burst release. The in vitro bioactivity of rhBMP2 released from PUR scaffolds was comparable to that of fresh rhBMP2, thereby demonstrating that this approach produced sustained release of active rhBMP2 over a 20-day period. To investigate the effects of release kinetics on new bone formation in vivo, PUR scaffolds incorporating rhBMP2 were implanted in 6-mm segmental femoral defects in Sprague-Dawley rats. A representative μCT image of a PUR/BMP2 scaffold at 4 weeks implantation time is shown in FIG. 33B. After both 4 and 8 weeks implantation time, PUR/BMP2 scaffolds exhibited significantly more new bone formation compared to the collagen+rhBMP2 control (FIG. 33C). However, PUR scaffolds with no rhBMP2 and PUR/PLGA-S-BMP2 scaffolds (slow release) showed only minimal new bone formation. These results suggest that both a burst and sustained release of rhBMP2 are desirable for new bone formation, which is consistent with our previous study in a rat femoral plug model. These embodiments show that the PUR scaffold is an effective carrier for rhBMP2 and has tunable release kinetics.

Dual delivery of vancomycin and rhBMP2 enhances new bone formation in an infected rat femoral segmental defect model. In non-infected rat calvarial defects, the activity of rhBMP2 released from a scaffold was reported to be unaffected by local delivery of the antibiotic teicoplanin. However, infection inhibits new bone formation, even in the presence of an osteogenic factor. While systemic delivery of antibiotics and local delivery of rhOP1 from a collagen carrier have been observed to enhance bone formation relative to local delivery of rhOP1 alone, the effects of local delivery of antibiotics and an osteoinductive factor on new bone formation in infected wounds have not been reported. To investigate the potential of local antibiotic delivery for protecting the implant from infection, we incorporated vancomycin free base (V-FB, 7 wt %) and rhBMP2 (60 or 600 μg/ml scaffold) as labile powders in PUR scaffolds. Collagen loaded with rhBMP2 was included in the study as a clinical control. Incorporation of V-FB and rhBMP2 significantly increased new bone formation and decreased the number of rats with clinical signs of infection. For example, 25-40% of the animals with only low doses of rhBMP2 exhibited clinical signs of infection, compared to 0-7% of the animals implanted with scaffolds incorporating rhBMP2 and V-FB. Few of the collagen or PUR scaffolds with the low (60 μg/ml) dose of rhBMP2 resulted in unions after 8 weeks in the presence of infection (FIG. 34(A), (C)), while nearly all animals exhibited unions at this rhBMP2 dose in uncontaminated defects at 4 weeks. Even at an order of magnitude higher dosage, many of the defects had not formed unions in the contaminated defect model (FIG. 34(B), (D)). However, when V-FB was incorporated in the PUR scaffolds, most of the animals had formed unions after 8 weeks, and the amount of new bone formation increased at the higher rhBMP2 dosage (FIG. 34(E), (F)). These data suggest that local dual delivery of vancomycin and rhBMP2 protects the implant from infection, allowing healing to proceed unimpeded. Thus embodiments of the present invention are effective dual-delivery devices.

List of acronyms, abbreviations, and symbols that may have been used in the examples or specification:
BVF bone void filler
CeMBR Center for Military Biomaterials Research
cGLP certified Good Laboratory Practice
cGMP certified Good Manufacturing Practice
HPLC High-Performance Liquid Chromatography
ISO International Organization for Standardization
LTI lysine triisocyanate
LTI-PEG lysine triisocyanate-poly(ethylene glycol) prepolymer
MDA methylene diphenyl diamine
MDI methylene diphenyl diisocyanate
OEF Operation Enduring Freedom
OETRP Orthopaedic Extremity Trauma Research Program
OIF Operation Iraqi Freedom
PEG poly(ethylene glycol)
PLGA poly(DL-lactide-co-glycolide)
Plexur™ LV Plexur Low Viscosity
PMMA poly(methyl methacrylate)
PUR polyurethane
rhBMP2 recombinant human BMP-2
V-FB vancomycin free base

Below are additional/supplemental examples of common acronyms used herein:
PUR Polyurethane
PDGF-BB Platelet-derived growth factor-BB
BMP-2 Bone morphogenetic protein-2
PLGA Poly (lactic-co-glycolic acid)
T Tobramycin
PEG Poly(ethylene glycol)
GF Growth factor
PUR/PDGF-BB Polyurethane/Platelet-derived growth factor-BB composite delivery system
PUR/G-PDGF-BB Polyurethane/gelatin coated PLGA Granule/Platelet-derived growth factor-BB composite delivery system
PUR/BMP-2 Polyurethane/Bone morphogenetic protein-2 composite delivery system
PUR/PLGA-L-BMP-2 Polyurethane/Poly (lactic-co-glycolic acid) large particle/Bone morphogenetic protein-2 composite delivery system
PUR/PLGA-S-BMP-2 Polyurethane/Poly (lactic-co-glycolic acid) small particle/Bone morphogenetic protein-2 composite delivery system
PUR/T/PLGA-L-BMP-2 Polyurethane/Tobramycin/Poly (lactic-co-glycolic acid) large particle/Bone morphogenetic protein-2 composite delivery system
PUR/T/PLGA-S-BMP-2 Polyurethane/Tobramycin/Poly (lactic-co-glycolic acid) small particle/Bone morphogenetic protein-2 composite delivery system
BSA-FITC Bone serum albumin-fluorescein isothiocyanate
PUR/BSA-FITC Polyurethane/Bone serum albumin-fluorescein isothiocyanate composite delivery system

The invention thus being described, it will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the Specification, including the Example, be considered as exemplary only, and not intended to limit the scope and spirit of the invention.

Unless otherwise indicated, all numbers expressing quantities of ingredients, properties such as reaction conditions, and so forth used herein are to be understood as being modified in all instances by the term “about.” Accordingly, unless indicated to the contrary, the numerical parameters set forth in the herein are approximations that may vary depending upon the desired properties sought to be determined by the present invention.

Notwithstanding that the numerical ranges and parameters setting forth the broad scope of the invention are approximations, the numerical values set forth in the experimental or example sections are reported as precisely as possible. Any numerical value, however, inherently contain certain errors necessarily resulting from the standard deviation found in their respective testing measurements.

Throughout this application, various publications are referenced. All such references, specifically including the three lists below, are incorporated herein by reference.

### REFERENCES

1. R. B. Gustilo and J. T. Anderson, Journal of Bone & Joint Surgery, American volume, 84, 682 (2002).

2. R. B. Gustilo, R. L. Merkow and D. Templeman, Journal of Bone & Joint Surgery, 72, 299-304 (1990).

3. C. M. Kelly, R. M. Wilkins, S. Gitelis, C. Hartjen, J. T. Watson and P. T. Kim, Clinical Orthopaedics & Related Research, 382, 42-50 (2001).

4. W. W. Rittmann and S. M. Perren, Cortical bone healing after internal fixation and infection, Springer, New York (1974).

5. P. Worlock, R. Slack, L. Harvey and R. Mawhinney, Injury, 25, 31-38 (1994).

6. A. A. Beardmore, D. E. Brooks, J. C. Wenke and D. B. Thomas, Journal of Bone & Joint Surgery, 87A, 107-112 (2005).

7. J. Calhoun and J. Mader, Clinical Orthopaedics & Related Research, 341, 206-214 (1997).

8. L. Dahners and C. Funderburk, Clinical Orthopaedics & Related Research, 219, 278-282 (1987).

9. J. Humphrey, S. Mehta, A. Seaber and T. Vail, Clinical Orthopaedics & Related Research, 349, 218-224 (1998).

10. K. Kanellakopoulou and E. J. Giamarellos-Bourboulis, Drugs, 59, 1223-1232 (2000).

11. J. T. Mader, J. Calhoun and J. Cobos, Antimicrobial Agents and Chemotherapy, 41, 415-418 (1997).

12. J. Rogers-Foy, D. Powers, D. Brosnan, S. Barefoot, R. Friedman and M. LaBerge, Journal of Investigative Surgery, 12, 263-275 (1999).

13. H. Buchholz, R. Elson and K. Heinert, Clinical Orthopaedics & Related Research, 190, 96-108 (1984).

14. P. Osterman, D. Seligson and S. Henry, Journal of Bone & Joint Surgery, British volume, 77, 93-97 (1995).

15. S. K. Seeley, J. V. Seeley, P. Telehowski, S. Martin, M. Tavakoli, S. L. Colton, B. Larson, P. Forrester and P. J. Atkinson, Clinical Orthopaedics & Related Research, 420, 298-303 (2004).

16. C. M. Stevens, K. D. Tetsworth, J. H. Calhoun and J. T. Mader, Journal of Orthopaedic Research, 23, 27-33 (2005).

17. S. Gitelis and G. Brebach, Journal of Orthopaedic Surgery (Hong Kong), 10, 53-60 (2002).

18. M. McKee, L. Wild, E. Schemitsch and J. Waddell, Journal of Orthopaedic Trauma, 16, 622-627 (2002).

19. A. C. McLaren, Clinical Orthopaedics & Related Research, 427, 101-106 (2004).

20. C. G. Ambrose, G. R. Gogola, T. A. Clyburn, K. A. Raymond, A. S. Peng and A. G. Mikos, Clinical Orthopaedics & Related Research, 415, 279-285 (2003).

21. S. Gogolewski and K. Gorna, Journal of Biomedical Materials Research Part A, 80A, 94-101 (2007).

22. J. Guan, K. L. Fujimoto, M. S. Sacks and W. R. Wagner, Biomaterials, 26, 3961-3971 (2005).

23. S. Guelcher, A. Srinivasan, A. Hafeman, K. Gallagher, J. Doctor, S. Khetan, S. McBride and J. Hollinger, Tissue Engineering, 13, 2321-2333 (2007).

24. S. A. Guelcher, V. Patel, K. M. Gallagher, S. Connolly, J. E. Didier, J. S. Doctor and J. O. Hollinger, Tissue Engineering, 12, 1247-1259 (2006).

25. A. E. Hafeman, B. Li, T. Yoshii, K. Zienkiewicz, J. M. Davidson and S. A. Guelcher, Pharmaceutical Research, In press (2008).

26. J. Guan, J. J. Stankus and W. R. Wagner, Journal of Controlled Release, 120, 70-78 (2007).

27. R. Adhikari and P. A. Gunatillake, Biodegradable polyurethane/urea compositions, U.S. Pat. Nos. 20,050, 238,683 (2004).

28. A. S. Sawhney and J. A. Hubbell, Journal of Biomedical Materials Research, 24, 1397-1411 (1990).

29. ASTM-International, D3574-05. Standard test methods for flexible cellular materials—slab, bonded, and molded urethane foams, pp 360-368 (2007).

30. G. Oertel, Polyurethane Handbook, Hanser Gardner Publications, Berlin (1994).

31. M. C. Caturla, E. Cusido and D. Westerlund, Journal of Chromatography A, 593, 69-72 (1992).

32. ASTM-International, D695-02a. Standard Test Method for Compressive Properties of Rigid Plastics (2007).

33. J. E. Mark, E. Erman and F. R. Eirich, Eds. Science and Technology of Rubber, Academic Press, Inc., San Diego, Calif. (1994).

34. C. G. Ambrose, T. A. Clyburn, K. Louden, J. Joseph, J. Wright, P. Gulati, G. R. Gogola and A. G. Mikos, Clinical Orthopaedics & Related Research, 421, 293-299 (2004).

35. M. Hombreiro-Perez, J. Siepmann, C. Zinutti, A. Lamprecht, N. Ubrich, M. Hoffman, R. Bodmeier and P. Maincent, Journal of Controlled Release, 88, 413-428 (2003).

36. J.-Y. Zhang, B. A. Doll, E. J. Beckman and J. O. Hollinger, Tissue Engineering, 9, 1143-1157 (2003).

37. Chen X, et al. J Orthop Res. 2005; 23 (4):816-23.

38. Gogolewski S, Gorna K, Turner AS. J Biomed Mater Res A. 2006; 77A (4):802-10.

39. Ambrose C G, et al. Clin Orthop Relat Res. 2003; 415:279-85.
1. Owens B D, Kragh J F, Jr., Macaitis J, Svoboda S J, Wenke J C. Characterization of extremity wounds in Operation Iraqi Freedom and Operation Enduring Freedom. J Orthop Trauma 2007; 21 (4):254-7.
2. Johnson E N, Burns T C, Hayda R A, Hospenthal D R, Murray C K. Infectious complications of open type III tibial fractures among combat casualties. Clinical Infectious Diseases 2007; 45:409-15.
3. Harris A M, Althausen P L, Kellam J, Bosse M J, Castillo R. Complications following limb-threatening lower extremity trauma. J Orthop Trauma 2009; 23 (1):1-6.
4. Hafeman A, Li B, Yoshii T, Zienkiewicz K, Davidson J, Guelcher S. Injectable biodegradable polyurethane scaffolds with release of platelet-derived growth factor for tissue repair and regeneration. Pharm Res 2008; 25 (10):2387-99.
5. Guelcher S, Srinivasan A, Hafeman A, Gallagher K, Doctor J, Khetan S, McBride S, Hollinger J. Synthesis, In vitro degradation, and mechanical properties of two-component poly(ester urethane)urea scaffolds: Effects of water and polyol composition. Tissue Engineering 2007; 13 (9):2321-2333.
6. Zhang J-Y, Beckman E J, Piesco N J, Agarwal S. A new peptide-based urethane polymer: synthesis, biodegradation, and potential to support cell growth in vitro. Biomaterials 2000; 21:1247-1258.
7. Bosse M J, MacKenzie E J, Kellam J F, Burgess A R, Webb L X, Swiontkowski M F, Sanders R W, Jones A L, McAndrew M P, Patterson B M and others. An analysis of outcomes of reconstruction or amputation after leg-threatening injuries. N Engl J Med 2002; 347 (24): 1924-31.
8. Friedman C D, Constantino P D, Takagi S, Chow L C. BoneSource™ hydroxyapatite cement: a novel biomaterial for craniofacial skeletal yissue engineering and reconstruction. J Biomed Mater Res 1998; 43 (4):428-32.
9. Chim H, Gosain A K. Biomaterials in craniofacial surgery experimental studies and clinical application J Craniofac Surg 2009; 20:29-33.
10. Gasparini G, Boniello R, Moro A, Tamburrini G, Di Rocco C, Febo S. Cranial reshaping using methyl methacrylate:technical note. J Craniofac Surg 2009; 20:184-190.
Moreira-Gonzalez A, Jackson I T, Miyawaki T, Barakat K, DiNick V. Clinical outcome in cranioplasty: critical review in long-term follow-up. J Craniofac Surg 2003; 14:144-153.
12. Bohner M. Calcium orthophosphates in medicine: from ceramics to calcium phosphate cements. Injury 2000; 31 (Supplement 4):S-D37-47.
13. Hollier L H, Stal S. The use of hydroxyapatite cements in craniofacial surgery. Clin Plastic Surg 2004; 31:423-428.
14. United States Food and Drug Administration Center for Devices and Radiological Health, Manufacturer and User Facility Device Experience Database. Available at http://www.fda.gov/cdrh/maude.html. Accessed Nov. 5, 2008.
15. Baker S B, Weinzweig J, Kirschner R E, Bartlett S P. Applications of a new carbonated calcium phosphate bone cement: early experience in pediatric and adult craniofacial reconstruction. Plast Reconstr Surg 2002; 109:1789-1796.
16. Goldberg C S, Antonyshyn O, Midha R, et al. Measuring pulsatile forces on the human cranium: J Craniofac Surg 2005; 16 (1):134-9.
17. Boyce T M, Winterbottom J M, Kaes D R, Shimp L A, Knaack D A. Development of a bone-derived composite implant for skeletal applications. In: Hollinger J O, editor; 2003; Pittsburgh, Pa.
18. Osteotech I. Concerned about the safety of your autograft?; 2006.
19. Bilezikian J P, Raisz L G, Rodan G A, editors. Principles of Bone Biology. Volume One. New York: Academic Press; 1996. 1-1398 p.
20. Market Dynamics:Bone Substitutes and Growth Factors. New York: Datamonitor (DMHC1812); 2002. p 118-32.
21. Newton C D, Nunamaker D B. Textbook of small animal orthopaedics. Philadelphia: Lippincott; 1985.
22. Adhikari R, Gunatillake P A, Griffiths I, Tatai L, Wickramaratna M, Houshyar S, Moore T, Mayadunne R T, Field J, McGee M and others. Biodegradable injectable polyurethanes: synthesis and evaluation for orthopaedic applications. Biomaterials 2008; 29 (28):3762-70.
23. Malinin T I, Carpenter E M, Temple H T. Particulate bone allograft incorporation in regeneration of osseous defects; importance of particle sizes. Open Orthop J 2007; 1:19-24.
24. Guelcher S. Biodegradable polyurethanes: synthesis and applications in regenerative medicine. Tissue Eng B 2008; 14 (1):3-17.
25. Storey R F, Wiggins J S, Puckett A D. Hydrolyzable poly(ester-urethane) networks from L-lysine diisocyanate and D,L-lactide/e-caprolactone homo- and copolyester triols. Journal of Polymer Science, Part A: Polymer Chemistry 1994; 32 (12):2345-2363.
26. Skarja G A, Woodhouse K A. Structure-property relationships of degradable polyurethane elastomers containing an amino acid-based chain extender. Journal of Applied Polymer Science 2000; 75:1522-1534.
27. Goma K, Gogolewski S. Preparation, degradation, and calcification of biodegradable polyurethane foams for bone graft substitutes. J Biomed Mater Res 2003; 67A:813-827.
28. Vertenten G, Vlaminck L, Gorski T, Schreurs E, Van Den Broeck W, Duchateau L, Schacht E, Gasthuys F. Evaluation of an injectable, photopolymerizable threedimensional scaffold based on D,L-lactide and e-caprolactone in a tibial goat model. J Mater Sci: Mater Med 2008; 19:2761-9.
29. Bennett S, Connolly K, Lee DR, Jiang Y, Buck D, Hollinger J O, Gruskin E A. Initial biocompatibility studies of a novel degradable polymeric bone substitute that hardens in situ. Bone 1996; 19 (1, Supplement):101S-107S.
30. Coury A. Chemical and Biochemical Degradation of Polymers. In: Ratner B, Hoffman A, Schoen F, Lemons J, editors. Biomaterials Science: An Introduction to Materials in Medicine. Boston: Elsevier Academic Press; 2004. p 411-430.
31. Blais P. Letter to the editor. J Appl Biomater 1990; 1:197.
32. Szycher M, Siciliano A. An assessment of 2,4-TDA formation from Surgitek polyurethane foam under stimulated physiological conditions. J Biomater Appl 1991; 5:323-336.
33. Chen X, Schmidt A H, Tsukayama D T, Bourgeault C A, Lew W D. Recombinant Human Osteogenic Protein-1 Induces Bone Formation in a Chronically Infected, Internally Stabilized Segmental Defect in the Rat Femur. J Bone Joint Sur Am 2006; 88:1510-1523.
34. Chen X Q, Kidder L S, Lew W D. Osteogenic protein-1 induced bone formation in an infected segmental defect in the rat femur. J Orthopaedic Res 2002; 20:142-150.
35. Hasegawa S, Ishii S, Tamura J, Furukawa T, Neo M, Matsusue M, Shikinami Y, Okuno M, Nakamura T. A 5-7 year in vivo study of high-strength hydroxyapatite/poly(L-lactide) composite rods for the internal fixation of bone fractures. Biomaterials 2066; 27:1327-1332.
36. Jeon O, Song S J, Kang S W, Putnam A J, Kim B S. Enhancement of ectopic bone formation by bone morphogenetic protein-2 released from a heparin-conjugated poly(L-lactic-co-glycolic acid) scaffold. Biomaterials 2007; 28 (17):2763-2771.
37. Jeon O, Kang S W, Lim H W, Chung J H, Kim B S. Long-term and zero-order release of basic fibroblast growth factor from heparin-conjugated poly(L-lactide-co-glycolide) nanospheres and fibrin gel. Biomaterials 2006; 27 (8):1598-1607.
38. Kempen D H, Kruyt M C, Lu L, Wilson C E, Florschutz A V, Creemers L B, Yaszemski M J, Dhert W J. Effect of Autologous BMSCs Seeding and BMP-2 Delivery on Ectopic Bone Formation in a Microsphere/Poly(Propylene Fumarate) Composite. Tissue Eng Part A 2008; 15 (3):587-94.
39. Kim S S, Gwak S J, Kim B S. Orthotopic bone formation by implantation of apatite-coated poly(lactide-co-glycolide)/hydroxyapatite composite particulates and bone morphogenetic protein-2. J Biomed Mater Res A 2008; 87 (1):245-53.
40. Li B, Yoshii T, Hafeman A E, Nyman J S, Wenke J C, Guelcher S A. The effects of rhBMP-2 released from biodegradable polyurethane/microsphere composite scaffolds on new bone formation in rat femora. Biomaterials 2009; 30 (35):6768-79.
41. Suzuki A, Terai H, Toyoda H, Namikawa T, Yokota Y, Tsunoda T, Takaoka K. A Biodegradable Delivery System for Antibiotics and Recombinant Human Bone Morphogenetic Protein-2: A Potential Treatment for Infected Bone Defects. J Orthop Res 2006; 24 (327-332).
42. Dumas J E, Zienkiewicz K L, Tanner S A, Prieto E M, Bhattacharyya S, Guelcher S A. Synthesis and Characterization of an Injectable Allograft Bone/polymer Composite Bone Void Filler with Tunable Mechanical Properties. Tissue Eng Submitted 2009.
43. Beckwith A C, Paulis J W, Wall J S. Direct estimation of lysine in corn meals by the ninhydrin color reaction. Journal of Agriculture and Food Chemistry 1975; 23 (2):194-196.
1. PCT Application PCT/US08/075481 published as WO2009033088 (A1) entitled
“Release of Antibiotic from Injectable, Biodegradable Polyurethane Scaffolds for Enhanced Bone Fracture Healing” filed Sep. 5, 2008. Inventors: Guelcher, Hafeman, Bing Li, K L Zienkiewicz
ABSTRACT: A biodegradable polyurethane scaffold, comprising at least one polyisocyante, polyisocyanate prepolymer, or both, at least one polyester polyol, at least one catalyst, wherein the density of said scaffold is from about 50 to about 250 kg m-3 and the porosity of the scaffold is greater than about 70 (vol %) and at least 50% of the pores are interconnected with another pore, and wherein the scaffold incorporates at least one biologically active component in powder form.
2. PCT Application PCT/US08/075498 published as W02009033102 (A1) entitled
“Polyurethane/Bone Composition and Methods” filed Sep. 5, 2008. Inventors: Guelcher, Dumas
ABSTRACT: A flowable, injectable composite that comprises mineralized allograft bone; and at least one degradable polyurethane that has a quasi-prepolymer and a resin mix, the resin mix having a polyester polyol and a catalyst; wherein the composite has a compression strength of greater than about 10 MPa and a modulus of greater than about 1 GPa.
3. US2009/0130174 A1 entitled “Poly(Ester Urethane)Urea Foams with Enhanced Mechanical and Biological Properties” filed Aug. 20, 2008. PCT Application PCTUS08/073754 entitled Poly(Ester Urethane)Urea Foams with Enhanced Mechanical and Biological Properties” filed Aug. 20, 2008. Inventors: Guelcher, Hafeman.
ABSTRACT: A biodegradable polyurethane scaffold that includes a HDI trimer polyisocyanate and at least one polyol; wherein the density of said scaffold is from about 50 to about 250 kg m⁻³ and the porosity of the scaffold is greater than about 70 vol % and at least 50% of the pores are interconnected with another pore. The scaffolds of the present invention are injectable as polyurethane foams, and are useful in the field of tissue engineering.
4. U.S. Pat. No. 12/473,246 entitled “Injectable Bone/Polymer Composite Bone Void Fillers” filed May 27, 2009. Inventors: Guelcher, Hafeman.
ABSTRACT: An embodiment of the present invention is a biocompatible and biodegradable polymer-bone composition. Particularly, embodiments of the present invention include biocompatible and biodegradable polymeric foams. In several embodiments, these foams are injectable. Related embodiments of the present invention include methods and compositions for their preparation and the use of these foams as scaffolds for bone tissue engineering.
5. PCT/US2009/062621 “Bone/Polyurethane Composites and Methods Thereof” filed Oct. 29, 2009. Inventors: Guelcher, Tanner, Zienkiewicz.
ABSTRACT: The invention relates to injectable and/or moldable composites/compositions including at least bone particles and polyurethanes, methods of making such composites, methods of using such composites in orthopaedic applications and various related compositions. The present invention provides porous composites which, when implanted or injected, promote cellular infiltration from adjacent osseous tisses, thus accelerating the remodeling process. Inventive composites comprise bone particles and polymers, such as a biocompatible polyurethane, and may further comprise additional components. The present invention also provides compositions, methods and processes that can be used for the preparation of such composites. The invention also provides methods and kits for making and/or using such inventive porous materials.
6. VU0928 Invention Disclosure entitled “Allograft Bone/Polyurethane Composites Prepared from Triisocyanates and Prepolymers.” Inventors: Guelcher, Dumas, Zienkiewicz, Prieto.
ABSTRACT: A conformable, moldable composite that comprises mineralized allograft bone (greater than or equal to 50 vol %); and at least one degradable polyurethane that has a polyisocyanate, a polyester polyol, and a catalyst mix; wherein the composite has a compression strength of greater than about 5 MPa and a modulus of greater than about 200 MPa.

We claim:

1. A biodegradable polyurethane scaffold, comprising
at least one polyisocyante, polyisocyanate prepolymer, or both;
at least one polyester polyol;
at least one catalyst;
wherein the density of said scaffold is from about 50 to about 250 kg m-3 and the porosity of the scaffold is greater than about 70 (vol %) and at least 50% of the pores are interconnected with another pore; and
wherein the scaffold incorporates at least one biologically active component in powder form.

2. The scaffold of claim 1, wherein the biologically active component has a hydroxyl or amine group.

3. The scaffold of claim 1, wherein the biologically active component is chosen from antibiotics, proteins, anti-cancer agents.

4. The scaffold of claim 1, wherein the biologically active component is a antibiotic.

5. The scaffold of claim 1, wherein the biologically active component is in powder form and chosen from tobramycin, colistin, tigecycline, BSA, PDGF, BMP-2.

6. The scaffold of claim 4, wherein the antibiotic is a labile tobramycin powder.

7. The scaffold of claim 1, wherein the polyisocyante is an aliphatic polyisocyanates chosen from lysine methyl ester diisocyanate (LDI), lysine triisocyanate (LTI), 1,4-diisocyanatobutane (BDI), and hexamethylene diisocyanate (HDI), and dimers and trimers of HDI.

8. The scaffold of claim 1, wherein the density is at least about 90 kg m-3.

9. The scaffold of claim 1, further comprising an excipient in a range of 5 about wt % or less, or in a range of from about 0.5 wt % to about 4 wt %.

10. The polyurethane scaffold of claim 1, further comprising PEG.

11. The polyurethane scaffold of claim 10, wherein the PEG is present in an amount of about 50% or less w/w or in an amount of about 30% or less w/w.

12. The scaffold of claim 1, wherein the porosity is greater than 70 (vol-%), or the porosity is from about 90 to about 95 (vol-%).

13. The scaffold of claim 1, further comprising a stabilizer chosen from a polyethersiloxane, sulfonated caster oil, and sodium ricinoleicsulfonate.

14. The scaffold of claim 1, further comprising a second biologically active agent.

15. The scaffold of claim 14, wherein the second biologically active agent comprises demineralized bone particles.

16. The scaffold of claim 1, wherein the second biologically active agent is chosen from enzymes, organic catalysts, ribozymes, organometallics, proteins, glycoproteins, peptides, polyamino acids, antibodies, nucleic acids, steroidal molecules, antibiotics, antivirals, antimycotics, anticancer agents, analgesic agents, antirejection agents, immunosuppressants, cytokines, carbohydrates, oleophobics, lipids, extracellular matrix and/or its individual components, demineralized bone matrix, pharmaceuticals, chemotherapeutics, cells, viruses, virenos, virus vectors, and prions.

17. The scaffold of claim 1, wherein the biologically active agent is an antibiotic, and is present in an amount of from about 1-12 wt %.

18. The scaffold of claim 17, wherein the biologically active agent is present in an amount of from about 2 to about 10 wt %; or the biologically active agent is present in an amount of from 4 to about 10 wt %.

19. The scaffold of claim 1, wherein the biologically active agent is a protein, and is present in an amount of from about 0.01 to about 10000 μg/ml of scaffold; or in an amount from about 0.1 to about 5000 μg/ml of scaffold; or in an amount from about 1 to about 5000 μg/ml of scaffold.

20. A composition that comprises:
at least one polyisocyante, polyisocyanate prepolymer, or both;
at least one polyester polyol;
at least one catalyst; and
at least one biologically active component in powder form.

21. The composition of claim 20, wherein the biologically active component is chosen from antibiotics, proteins, anti-cancer agents.

22. The composition of claim 21, wherein the biologically active component is an antibiotic incorporated in powdered form.

23. The composition of claim 22, wherein the biologically active agent is a protein, and is present in an amount of from about 0.01 to about 10000 μg/ml of scaffold; or in an amount from about 0.1 to about 5000 μg/ml of scaffold or in an amount from about 1 to about 5000 μg/ml of scaffold.

24. The composition of claim 22, wherein the antibiotic is a labile tobramycin powder.

25. The composition of claim 20, further comprising an excipient in a range of 5 about wt % or less, or in a range of from about 0.5 wt % to about 4 wt %.

26. The composition of claim 20, further comprising a PEG in an amount of about 50% or less w/w or in an amount of about 30% or less w/w.

27. The composition of claim 20, further comprising a stabilizer chosen from a polyethersiloxane, sulfonated caster oil, and sodium ricinoleicsulfonate.

28. The composition of claim 20, wherein the biologically active agent is incorporated as a powder and is present in an amount of from about 2 to about 10 wt %; or the biologically active agent is present in an amount of from 4 to about 10 wt %.

29. The composition of claim 20, wherein the polyisocyante is an aliphatic polyisocyanates chosen from lysine methyl ester diisocyanate (LDI), lysine triisocyanate (LTI), 1,4-diisocyanatobutane (BDI), and hexamethylene diisocyanate (HDI), and dimers and trimers of HDI.

30. A method of delivering a biologically active agent to a wound site comprising:
providing a composition that comprises at least one polyisocyante, polyisocyanate prepolymer, or both; at least one polyester polyol; at least one catalyst; and at least one biologically active component in powder form; and
contacting the composition with a wound site.

31. The method of claim 30, wherein the wound site is a bone.

32. The method of claim 30, wherein the wound site is skin.
